(19) United States
(12) Patent Application Publication
(10) Pub. No.: US 2010/0310530 A1
(43) Pub. Date: Dec. 9, 2010

(54) Cell Therapy for Brain Tissue Damage

(75) Inventors: Shinn-Zong Lin, Taichung City (TW); Hung Li, Taipei City (TW); Woei-Cherng Shyu, Taipei City (TW); Hsiu-Mei Hsieh-Li, Taipei City (TW)

Correspondence Address: OCCHIUTI ROHLICEK & TSAO, LLP, 10 FAWCETT STREET, CAMBRIDGE, MA, 02138, US

(73) Assignee: StemCyte Inc., Ewing, NJ (US)

(21) Appl. No.: 12/784,538

(22) Filed: May 21, 2010

Related U.S. Application Data
(60) Provisional application No. 61/180,243, filed on May 21, 2009.

(51) Int. Cl. A61K 35/12

(52) U.S. Cl. 424/93.7

(57) ABSTRACT

Disclosed are methods for conditioning stems cells and using the conditioned stems cells for treating brain tissue damage.

### RELATED APPLICATION

This application claims priority to U.S. Provisional Application No. 61/180,243, filed on May 21, 2009, the content of which is hereby incorporated by reference in its entirety.

### BACKGROUND OF THE INVENTION

Brain tissue damage, resulting either from injuries or disorders (e.g., neurodegenerative and cerebrovascular diseases), are a leading cause of long-term disability. Due to their pluripotency, embryonic stem cells (ES cells) hold a great promise for treating brain tissue damage. However, ethical and logistical considerations have hampered their use. Use of non-ES pluripotent cells has been exploited. Nonetheless, such cells have limited neuroplasiticity. Thus, there is a need for a method for improving their neuroplasiticity.

### SUMMARY OF INVENTION

This invention is based, at least in part, on the unexpected finding that hypoxia preconditioning (HP) can be used to improve neuroplasiticity and differentiation capacity of non-ES pluripotent cells. Cells so improved can be used to treat brain tissue damage.

Accordingly, one aspect of this invention features a method of improving neurological behavior function of a subject having brain tissue damage. The method includes identifying a subject suffering from brain tissue damage, and administering to the subject a composition containing an effective amount of a pluripotent cell. The pluripotent cell can be any suitable stem cell such as an ES cells, hematopoietic stem cells (HSCs), or bone marrow stem cell. In one embodiment, the pluripotent cell is a CD34⁺ cell, such as a CD34⁺ cell and is obtained from umbilical cord blood. The process can further include evaluating the Epac1 level in the cell after culturing the cell under a hypoxia condition. The composition can be administered intracerebrally. In one embodiment, the method further includes a step of evaluating a therapeutic effect on the subject by a non-invasive technique.

The pluripotent cell is prepared by a process comprising culturing the cell under a hypoxia condition. Hypoxia condition (HP) refers to a condition which induces a sub-lethal stress in a cell, activates various endogenous trophic signals, and induces robust protection against subsequent lethal insults. It can be brought about by subjecting a cell to a short-term hypoxia or incubating the cell with certain chemical agents for a period of time. For example, culturing the cell under a hypoxia condition can be achieved conducted by placing the cell in a medium containing 60 to 600 mM Desferrioxamine (DFX) for 12 to 48 hours. In another, culturing the cell under a hypoxia condition is conducted by placing the cell in a medium containing 100 to 450 mM Desferrioxamine (DFX) for 16 to 36 hours. In yet another embodiment, it is conducted by placing the cell in a medium containing 200 to 350 mM Desferrioxamine (DFX) for 20 to 24 hours. Alternatively, one can incubate the cell in a medium containing CoCl₂ for 12 to 48 hours. The concentration of CoCl₂ can range from 10-500 μM. In a prefer embodiment, the CoCl₂ convention is about 100 μM.

Culturing the cell under a hypoxia condition can also be carried out under a condition where the oxygen level is lower than that in a normal cell culture condition for a period of time. For example, culturing the cell under a hypoxia condition can be conducted by placing the cell in an environment (e.g., an incubator) containing 0.5 to 3% O₂ for 6 to 48 hours, 0.8 to 1.5% O₂ for 12 to 36 hours, or 0.9 to 1.1% 0₂ for 23 to 25 hours.

In another aspect, this invention features a method of increasing angiogenesis in a tissue of a subject. The method includes administering to a tissue of a subject in need thereof a composition containing an effective amount of a pluripotent cell. The pluripotent cell is prepared in the same manner described above. In one example, the method can be used to increase angiogenesis in the brain of a subject having brain tissue damage.

### BRIEF DESCRIPTION OF DRAWINGS

FIG. 1A-1 and 1A-2 are photographs and diagrams showing results of Western blots.

FIGS. 2A to 2G2 are an illustration showing a treatment and neurological behavior measurement protocol (2A) and diagrams and photographs showing results of the treatment (2B-2G).

FIGS. 3A-1 to 3G-2 are photographs and diagrams showing angiogenesis caused by engraftments of stem cells in brains.

FIGS. 4A-1 to 4F are photographs and diagrams showing effects on Epac 1 or MMP2 expression by engraftments of stem cells in brains.

FIGS. 5A-1 to 5C-2 are photographs and diagrams showing neurogenesis caused by engraftments of stem cells in brains.

### DETAILED DESCRIPTION OF THE INVENTION

It has been suggested that ES cells can be used to regenerate neuronal or glial cells in the brain and thereby treat brain tissue damage. However, ethical and logistical considerations have hampered the use of ES cells. Non-ES pluripotent cells, such as bone marrow-derived mesenchymal stem cells (MSCs) and human umbilical cord blood (hUCB), represent a promising alternative. However, these alternatives are not always acceptable due to the significant decreases in cell number and proliferation/differentiation capacity with age.

Human umbilical cord blood, due to its primitive nature and ease of collection, appears to be a promising candidate for multipotent stem cell harvest, and could offer an interesting alternative for cellular therapies applied to brain regeneration. Particularly, population by selection for CD34, a surface molecule expressed on progenitors from hematopoietic, endothelial, and neural lineages, hided rich in hUCB and contained a higher number of early progenitor cells. However, few have investigated the hematopoietic stem cells (HSCs) that regulate the fate of endogenous NSCs. Also, because the microenvironment in the host brain is “toxic” to the implanted stem cells, many implanted cells die soon after transplantation (Wei et al., 2005, Neurobiol Dis 19:183-193). While several strategies have been explored to enhance the engraftment of transplanted cells and increase the therapeutic potential of transplantation therapy (Wei et al., 2005, Neurobiol Dis 19:183-193; Chen et al., 2002, J Neurol Sci 199:17-24; and Park et al., 2003, Neurosci Lett 353:91-94), there are still many limitations associated with each of the approaches, mostly rendering them clinically unfeasible at the current time.

As described herein, it was unexpected that hypoxia preconditioning can be used to improve neuroplasiticity and differentiation capacity of non-ES pluripotent cells. Hypoxic preconditioning (HP) is a sub-lethal stress induced by short-term hypoxia that activates various endogenous trophic signals and induces robust protection against subsequent lethal insults (Kirino et al., 2002, J Cereb Blood Flow Metab 22:1283-1296 and Gidday, 2006, Nat Rev Neurosci 7:437-448). As described herein, it represents a tool with which to identify new therapeutic targets against ischemic damage. Some have investigated the therapeutic potential of using HP-MSCs, but there was little success (Danet et al., 2003, J Clin Invest 112:126-135).

As described herein, it was discovered that HP could upregulate the expression of Exchange protein activated by cAMP-1 (Epac1) via HIF-1α activation, and then increase the Rap1-GTP activity. It was also found that intracerebral HP-hUCB derived HSCs (HP-hUCB³⁴) implantation enhanced the neuroplasticity in the cerebral ischemic model through promoting neurite outgrowth and MMP secretion by the molecular mechanism of activation of Epac1-Rap1 signaling.

Epac1 are guanine nucleotide exchange factors for the small GTPase Rap1 and Rap2 (Bos, 2006, Trends Biochem Sci 31:680-686). Epac1 activation could enhance Rap1 activity to promote β1-integrin-mediated adhesion and increase matrix metalloprotease (MMP2/9) secretion. Recently, Epacl signaling was found to be related to the axonal regeneration. Activation of Epac1 promotes neurite outgrowth, which is as effective as cAMP elevation in enhancing neurite regeneration on spinal cord tissue. It was also shown that activated Epac1 acted synergistically with NGF to promote neurite extension in PC-12 rat pheochromocytoma cells. Furthermore, activation of Epac1 in the endothelial progenitor cells (EPCs) could increase EPCs homing to ischemic muscles and neovascularization in the model of hind limb ischemia.

The contribution of tissue hypoxia as a stimulus for the induction of Epac1 was not known. Due to metabolic regulation during hypoxia, the interstitial adenosine concentration rises to levels that activate endothelial adenosine receptors (ARs) and promote endothelial cell proliferation and migration.

The present invention relates to conditioning stem cells, such as umbilical cord blood stem cells (hUCB), under an HP condition. Stem cells that have been thus conditioned can be used to treat brain tissue damage. Various stem cells can be used in this invention. Examples of the stem cells include umbilical cord blood cells, hematopoietic stem cells, embryonic stem cells, and other stem cells that can differentiate into functional neuronal or glial cells.

The term “stem cell” refers to a cell that is capable of differentiating into a number of final, differentiated cell types. Stem cells may be totipotent or pluripotent. Totipotent stem cells typically have the capacity to develop into any cell type. Totipotent stem cells can be both embryonic and non-embryonic in origin. Pluripotent cells are typically cells capable of differentiating into several different, final differentiated cell types. Unipotent stem cells can produce only one cell type, but have the property of self-renewal which distinguishes them from non-stem cells. These stem cells can originate from various tissue or organ systems, including, but not limited to, blood, nerve, muscle, skin, gut, bone, kidney, liver, pancreas, thymus, and the like. In accordance with the present invention, the stem cell can be derived from an adult or neonatal tissue or organ.

The cells described in this invention are substantially pure. The term “substantially pure”, when used in reference to stem cells or cells derived therefrom (e.g., differentiated cells), means that the specified cells constitute a substantial portion of or the majority of cells in the preparation (i.e., more than 20%, 30%, 40%, 50%, 60%, 70%, 80%, 90%, or 95%). Generally, a substantially purified population of cells constitutes at least about 70% of the cells in a preparation, usually about 80% of the cells in a preparation, and particularly at least about 90% of the cells in a preparation (e.g., 95%, 97%, 99% or 100%).

In a preferred embodiment, umbilical cord blood cells are used. These stem cells can be enriched by methods known in the art and then tested by standard techniques. To confirm the differentiation potential of the cells, they can be induced to form, for example, various colony forming units, by methods known in the art.

The cells thus confirmed can be further propagated in a non-differentiating medium culture for more than 10, 20, 50, or 100 population doublings without indications of spontaneous differentiation, senescence, morphological changes, increased growth rate, or changes in ability to differentiate into neurons. The cells can be stored by standard methods before use.

The terms “proliferation” and “expansion” as used interchangeably herein with reference to cells, refer to an increase in the number of cells of the same type by division. The term “differentiation” refers to a developmental process whereby cells become specialized for a particular function, for example, where cells acquire one or more morphological characteristics and/or functions different from that of the initial cell type. The term “differentiation” includes both lineage commitment and terminal differentiation processes. Differentiation may be assessed, for example, by monitoring the presence or absence of lineage markers, using immunohistochemistry or other procedures known to a worker skilled in the art. Differentiated progeny cells derived from progenitor cells may be, but are not necessarily, related to the same germ layer or tissue as the source tissue of the stem cells. For example, neural progenitor cells and muscle progenitor cells can differentiate into hematopoietic cell lineages. The terms “lineage commitment” and “specification,” as used interchangeably herein, refer to the process a stem cell undergoes in which the stem cell gives rise to a progenitor cell committed to forming a particular limited range of differentiated cell types. Committed progenitor cells are often capable of self-renewal or cell division. The term “terminal differentiation” refers to the final differentiation of a cell into a mature, fully differentiated cell. For example, hematopoietic progenitor cells and muscle progenitor cells can differentiate into neural or glial cell lineages, terminal differentiation of which leads to mature neurons or glial cells. Usually, terminal differentiation is associated with withdrawal from the cell cycle and cessation of proliferation. The term “progenitor cell,” as used herein, refers to a cell that is committed to a particular cell lineage and which gives rise to cells of this lineage by a series of cell divisions.

Like ES cells, the conditioned hUCB possess potentials to differentiate into various cells, including neuronal cells or glial cells. They therefore can be used to regenerate the cells for treating brain tissue damage. As shown in the example below, hUCB can be easily isolated, maintained and expanded in vitro, and induced to differentiation using routine technical approaches. In addition, after grafting conditioned hUCB into mice or rats, there is no evidence of mitotically active cells, teratomas, or malignant growth. These cells can be used for transplantation in treating stroke, head injury, or neurodegeneration without the above-mentioned concerns. Due to these advantages, the cells represent an alternative to other pluripotent cells. The cells thus conditioned can be stored by standard methods or can be administered intracerebrally to a subject in need thereof.

Within the scope of this invention is a method of treating brain tissue damage or alleviate the symptom of the disorder in a subject. The method includes identifying a subject suffering from or being at risk for developing brain tissue damage. The subject can be a human or a non-human mammal, such as a cat, a dog, or a horse. Examples of the brain tissue damage includes those caused by a cerebral ischemia (e.g., chronic stroke) or a neurodegenerative disease (e.g., Parkinson's disease, Alzheimer's disease, Spinocerebellar disease, or Huntington's disease). A subject to be treated can be identified by standard techniques for diagnosing the conditions or disorders of interest. The treatment method entails administering to a subject in need thereof an effective amount of the above-described HP conditioned stem cells.

The therapeutic effects of the above-described cells can be accessed according to standard methods (e.g., those described in the example below). To confirm efficacy in promoting cerebrovascular angiogenesis, one can examine the subject before and after the treatment by standard brain imaging techniques, such as computed tomography (CT), Doppler ultrasound imaging (DUI), magnetic resonance imaging (MRI), and proton magnetic resonance spectroscopy (¹H-MRS). For example, ¹H-MRS represents a non-invasive means to obtain biochemical information correlated to brain metabolic activity (Lu et al., 1997, Magn. Reson. Med. 37, 18-23). This technique can be applied to evaluate the metabolic changes involved in cerebral ischemia with or without stem cell transplantation. For example, it can be used to study the N-acetylaspartate (NAA) concentration in the brain, a marker of neuronal integrity. Although NAA redistribution and trapping in neuronal debris limits its use as a quantitative neuronal marker, decreases in brain NAA concentration in cerebral ischemia can be considered as an index of neuronal loss or dysfunction (Demougeot et al., 2004, J. Neurochem. 90, 776-83). Therefore, an NAA level, measured by ¹H-MRS, is a useful indicator for following the effect of stem cell transplantation after cerebral ischemia.

One can also measure the expression level of a trophic factor or a cell death-related protein (e.g., Epac1 or MMP2) in a sample (e.g., cerebrospinal fluid) obtained from the animal before or after administration to confirm efficacy. The expression level can be determined at either the mRNA level or the protein level. Methods of measuring mRNA levels in a tissue sample or a body fluid are well known in the art. To measure mRNA levels, cells can be lysed and the levels of mRNA in the lysates, whether purified or not, can be determined by, e.g., hybridization assays (using detectably labeled gene-specific DNA or RNA probes) and quantitative or semi-quantitative RT-PCR (using appropriate gene-specific primers). Alternatively, quantitative or semi-quantitative in situ hybridization assays can be carried out on tissue sections or unlysed cell suspensions using detectably (e.g., fluorescent or enzyme) labeled DNA or RNA probes. Additional mRNA-quantifying methods include the RNA protection assay (RPA) method and the serial analysis of gene expression (SAGE) method, as well as array-based technologies.

Methods of measuring protein levels in a tissue sample or a body fluid are well known in the art. Some of them employ antibodies (e.g., monoclonal or polyclonal antibodies) that bind specifically to a target protein. In such assays, the antibody itself or a secondary antibody that binds to it can be detectably labeled. Alternatively, the antibody can be conjugated with biotin. Its presence can be determined by detectably labeled avidin (a polypeptide that binds to biotin). Combinations of these approaches (including “multi-layer sandwich” assays) can be used to enhance the sensitivity of the methodologies. Some protein-measuring assays (e.g., ELISA or Western blot) can be applied to body fluids or to lysates of cells, and others (e.g., immunohistological methods or fluorescence flow cytometry) can be applied to histological sections or unlysed cell suspensions. Appropriate labels include radionuclides (e.g., ¹²⁵I, ¹³¹I, ³⁵I, ³H, or ³²P), enzymes (e.g., alkaline phosphatase, horseradish peroxidase, luciferase, or β-glactosidase), fluorescent/luminescent agents (e.g., fluorescein, rhodamine, phycoerythrin, GFP, BFP, and Qdot™ nanoparticles supplied by the Quantum Dot Corporation, Palo Alto, Calif.). Other applicable methods include quantitative immunoprecipitation or complement fixation assays.

Based on the results from the assays described above, an appropriate dosage range and administration route can be determined. The dosage required depends on the choice of the route of administration; the nature of the formulation; the nature of the patient's illness; the subject's size, weight, surface area, age, and sex; other drugs being administered; and the judgment of the attending physician. Dosage variations are necessary in view of the different efficiencies of various routes of administration. The variations can be adjusted using standard empirical routines for optimization as is well understood in the art. In general, 1×10⁴ and 1×10⁷ (e.g., 1×10⁵ to 5×10⁶ and more preferably 5×10⁵ to 2×10⁵) cells are administered. Multiple sites can be used depending on the site and nature of particular damage. The example below describes approximate coordinates for administering cells in a rat ischemia model. Coordinates for other disorders in other species can be determined accordingly based on comparative anatomy.

Both heterologous and autologous hUCB can be used. In the former case, HLA-matching should be conducted to avoid or minimize host reactions. In the latter case, autologous hUCB are enriched and purified from a subject and stored for later use.

The invention also features a method of treating a neurodegenerative disease. The method includes identifying a subject suffering from or being at risk for developing a neurodegenerative disease, and administering to the subject an effective amount of pluripotent animal cells, which are processed in the manner described above. Examples of the neurodegenerative disease include Parkinson's disease, Alzheimer's disease, Spinocerebellar disease, or Huntington's disease. In all of the above-described methods, the cells are administered (e.g., intracerebrally) to a subject at 1×10⁴ to 1×10⁷/time, preferably at 1×10⁵ to 5×10⁶/time, or more preferably at 5×10⁵ to 2×10⁶/time. To minimize or avoid host rejections, the cells are preferably autologous to the subject.

The term “treating” refers to administration of a composition (e.g., a cell composition) to a subject, who is suffering from or is at risk for developing brain tissue damage or a disorder causing such damage, with the purpose to cure, alleviate, relieve, remedy, or ameliorate the damage/disorder, the symptom of the damage/disorder, the disease state secondary to the damage/disorder, or the predisposition toward the damage/disorder. The treatment method can be performed alone or in conjunction with other drugs or therapies.

The above-described methods may further include administering the subject with a minimal immunosuppressive regimen prior to, concomitantly with, or following transplantation of the cells. Various types of immunosuppressive regimens may be used. Examples include administration of immunosuppressive drugs, tolerance inducing cell populations, and/or immunosuppressive irradiation. Guidance for selecting and administering suitable immunosuppressive regimens for transplantation is well known in the art (e.g., Kirkpatrick et al., 1992. JAMA. 268, 2952; Higgins et al., 1996. Lancet 348, 1208; Suthanthiran et al., 1996. New Engl. J. Med. 331, 365; Midthun et al., 1997. Mayo Clin Proc. 72, 175; Morrison et al., 1994. Am J. Med. 97, 14; Hanto 1995 Annu Rev Med. 46, 381; Senderowicz et al., 1997. Ann Intern Med. 126, 882; Vincenti et al., 1998. New Engl. J. Med. 338, 161; Dantal et al. 1998. Lancet 351, 623).

Examples of suitable immunosuppressive drugs include CTLA4-Ig, anti-CD40 antibodies, anti-CD40 ligand antibodies, anti-B7 antibodies, anti-CD3 antibodies (for example, anti-human CD3 antibody OKT3), methotrexate (MTX), prednisone, methyl prednisolone, azathioprene, cyclosporin A (CsA), tacrolimus, cyclophosphamide and fludarabin, mycophenolate mofetil, daclizumab (a humanized (IgG1 Fc) anti-IL2R alpha chain (CD25) antibody), anti-T-lymphocyte antibodies conjugated to toxins (for example, cholera A chain, or Pseudomonas toxin), and an agent capable of inhibiting the activity of the protein mammalian-target-of-rapamycin (mTOR).

The present invention provides for pharmaceutical compositions containing the above-descried cells or active agents/compounds. In one example, the invention features a composition having the above-described pluripotent cell(s) (e.g., a CD34⁺ cell or one obtained from umbilical cord blood) and a hypoxia agent (e.g., Desferrioxamine (DFX) and CoCl₂. Pharmaceutical compositions can be prepared by mixing a therapeutically effective amount of the cells or active agents/compounds, and, optionally other active substance, with a pharmaceutically acceptable carrier. The carrier can have different forms, depending on the route of administration.

The above-described pharmaceutical compositions can be prepared by using conventional pharmaceutical excipients and methods of preparation. All excipients may be mixed with solvents, granulating agents, moisturizers, and binders. As used herein, the term “effective amount” or ‘therapeutically effective amount’ refers to an amount which results in measurable amelioration of at least one symptom or parameter of a specific disorder. A therapeutically effective amount of the above-descried cells can be determined by methods known in the art. An effective amount for treating a disorder can easily be determined by empirical methods known to those of ordinary skill in the art. The exact amount to be administered to a patient will vary depending on the state and severity of the disorder and the physical condition of the patient. A measurable amelioration of any symptom or parameter can be determined by a person skilled in the art or reported by the patient to the physician. It will be understood that any clinically or statistically significant attenuation or amelioration of any symptom or parameter of the above-described disorders is within the scope of the invention. Clinically significant attenuation or amelioration means perceptible to the patient and/or to the physician.

The phrase “pharmaceutically acceptable” refers to molecular entities and other ingredients of such compositions that are physiologically tolerable and do not typically produce unwanted reactions when administered to a human. Preferably, the term “pharmaceutically acceptable” means approved by a regulatory agency of the federal or a state government or listed in the U.S. Pharmacopeia or other generally recognized pharmacopeia for use in mammals, and more particularly in humans. Pharmaceutically acceptable salts, esters, amides, and prodrugs refers to those salts (e.g., carboxylate salts, amino acid addition salts), esters, amides, and prodrugs which are, within the scope of sound medical judgment, suitable for use in contact with the tissues of patients without undue toxicity, irritation, allergic response, and the like, commensurate with a reasonable benefit/risk ratio, and effective for their intended use.

A carrier applied to the pharmaceutical compositions described above refers to a diluent, excipient, or vehicle with which a compound is administered. Such pharmaceutical carriers can be sterile liquids, such as water and oils. Water or aqueous solution, saline solutions, and aqueous dextrose and glycerol solutions are preferably employed as carriers, particularly for injectable solutions. Suitable pharmaceutical carriers are described in “Remington's Pharmaceutical Sciences” by E. W. Martin, 18th Edition.

The above-descried cells can be administered to individuals through infusion or injection (for example, intravenous, intrathecal, intramuscular, intraluminal, intratracheal, intraperitoneal, or subcutaneous), orally, transdermally, or other methods known in the art. Administration may be once every two weeks, once a week, or more often, but frequency may be decreased during a maintenance phase of the disease or disorder.

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety. Further, any mechanism proposed below does not in any way restrict the scope of the claimed invention.

### Materials and Methods

### Purification and Selection of CD34⁺ hUCBs (hUCB³⁴)

Mononuclear cells (MNCs) were prepared from fresh or cryopreserved whole human umbilical cord blood (hUCB^{W}) (Stemcyte, USA) as previously described (Kekarainen et al., 2006, BMC Cell Biol 7:30). The MNC layer was collected using the Ficoll-Histopaque (Sigma, USA) centrifugation method (Asahara et al., 1997, Science 275:964-967), and washed twice with 1mM EDTA in PBS. The CD34⁺ MNCs were separated from 2×10⁸ MNCs by a magnetic bead separation method (MACS; Miltenyi Biotec, Gladbach, Germany) according to the manufacturer's instructions. In brief, MNCs were suspended in 300 μL, PBS and 5 mM EDTA. These cells were labeled with a hapten-conjugated mAb against CD34 (Miltenyi Biotec, Gladbach, Germany), followed by anti-hapten Ab coupled with microbeads, and were incubated with beads at ratios of 100 μL beads per 10⁸ cells for 15 minutes at 4° C. The bead-positive cells (CD34⁺ MNCs) were enriched on positive-selection-columns set in a magnetic field. FACS analysis using anti-CD34 antibodies (Miltenyi Biotec, Gladbach, Germany) labeled with phycoerythrin (PE) (Becton Dickinson, USA) of MACS-sorted cells showed that 94%±1.7% of the selected cells were positive for CD34 (data not shown). Then, cells were labeled with 1 μg/mL bis-benzimide (Hoechst 33342; Sigma, USA), and cultured for 72 hours in medium (StemSpan™ SFEM and Cytokine Cocktail, StemCell Technologies) at 37° C. in a humidified atmosphere of 5% CO₂/95% air and antibiotics, and prepared for further transplantation.

### Hypoxia Preconditioning (HP) Procedure and Phenotypic Analysis

hUCB³⁴ cells (1×10⁶/mL) were cultured in a StemSpan SFEM medium (StemCell Technologies, Vancouver, Canada) at 37° C. in 5% CO₂-humidified incubators in normoxic (20% O₂) or hypoxic (1% O₂) conditions as previously described (Ivanovic et al., 2000, Br J Haematol 108:424-429). Hypoxic cultures were cultivated in a two-gas incubator (Jouan, Winchester, Va., USA) equipped with an O₂ probe to regulate N₂ levels. Cell number and viability were evaluated using trypan blue exclusion assay. For flowcytometry, cells were incubated with anti-human CD34 (Miltenyi Biotec, Gladbach, Germany) and then analyzed on a FACSCalibur flow cytometer (Becton, Dickinson and Co.). To produce chemical hypoxia, cells were treated in a medium containing 60-600 mM of Desferrioxamine (DFX, Sigma-Aldrich, MO) that mimics hypoxic conditions by inhibiting the hydroxylation of a prolyl residue that is essential for the ubiquitination of HIF-1α (Schioppa et al., 2003, J Exp Med 198:1391-1402).

### Rap1 Activity Assay

Rap1 activation assays were performed using commercial Rap1-activity Assay Kit (Upstate) according to the manufacturer's instruction (Goichberg et al., 2006, Blood 107:870-879). In brief, hUCB³⁴ were treated with short-term hypoxia as mentioned above. Then cells were lysed in Rapl activation lysis buffer. Lysates were clarified by centrifugation, a portion of the cell lysate was reserved for analysis of total Rap1 content, and 500 μL of lysate was incubated with GST-tagged RBD of Ra1GDS pre-coupled to glutathione beads (Upstate) to specifically pull down the GTP-bound form of Rap1. Samples were incubated for 45 minutes at 4° C. with gentle rotation. Beads were washed 3 times in lysis buffer. Rap1 was detected using Western blot with antiRap1 antibodies (Upstate).

### Chromatin Immunoprecipitation (ChIP) Assay

To demonstrate the binding of HIF-1a protein to the Epacl promoter (Zanata et al., 2002, Embo J 21:3307-3316), ChIP assay was performed with a commercial kit (Upstate Biotechnology) using the manufacturer's protocol with minor adjustments. The hUCB³⁴ were grown and incubated in air or 1% O₂ for 4 h, and formaldehyde was added directly to the culture medium to a final concentration of 1% followed by incubation for 20 min at 37° C. as previously described (Ponnusamy et al., 2008, J Biol Chem 283:27514-27524). DNA-protein complexes were isolated on salmon sperm DNA linked to protein A agarose beads and eluted with 1% SDS, and 0.1 M NaHCO₃. Cross-linking was reversed by incubation at 65° C. for 5 h. Proteins were removed with proteinase K, and DNA extracted with phenol/chloroform, redissolved and PCR-amplified with Epac1 promoter primers, sense: 59-attcagcagatatagggcag-39; and antisense: 59-acagtcagctctcattaatg-39 (reverse).

### Electrophoretic Mobility Shift Assay (EMSA)

Detailed protocols to assess HIF-1α DNA binding activity using EMSA have been described previously (Yin et al., 2000, Biochem Biophys Res Commun 279:30-34). The nuclear extracts were prepared using a commercial kit (Pierce). An oligonucleotide probe (5′-CCTCCCGGCCACGTGGCGGCCAG-3′ and 5′-GGAGGGCCGGTGCA CCGCCGGTC-3′) corresponding to the hypoxia-response element (HRE) in the Epac1 gene promoter was used (Zanata et al., 2002, Embo J 21:3307-3316). The oligonucleotides were non-radioisotope labeled using Light-Shift Chemiluminiscent EMSA Kit (Pierce) under the manufacture's instruction. In brief, the binding reaction was performed in a reaction mixture of 20 mL that contained binding buffer (10 mM Tris-HCl, 20 mM NaCl, 1 mM DTT, 1 mM EDTA, and 5% glycerol, pH 7.6), 0.1 ng of labeled probe (>10,000 cpm), 30 μg of nuclear proteins, and 1 μg of poly(dI-dC). After incubation for 20 min at room temperature, the mixture was subjected to gel electrophoresis on a nondenaturing 6% polyacrylamide gel at 180V for 2-4 h under a low ionic strength condition. The gel was vaccum dried and subjected to autoradiography. For supershift assays, 1 82 g of anti-HIF-1α antibody (Novus Biologicals) was added to the samples 1 h prior to the addition of labeled probes.

### Experimental Animals Undergo Intracerebral hUCB³⁴ Transplantation

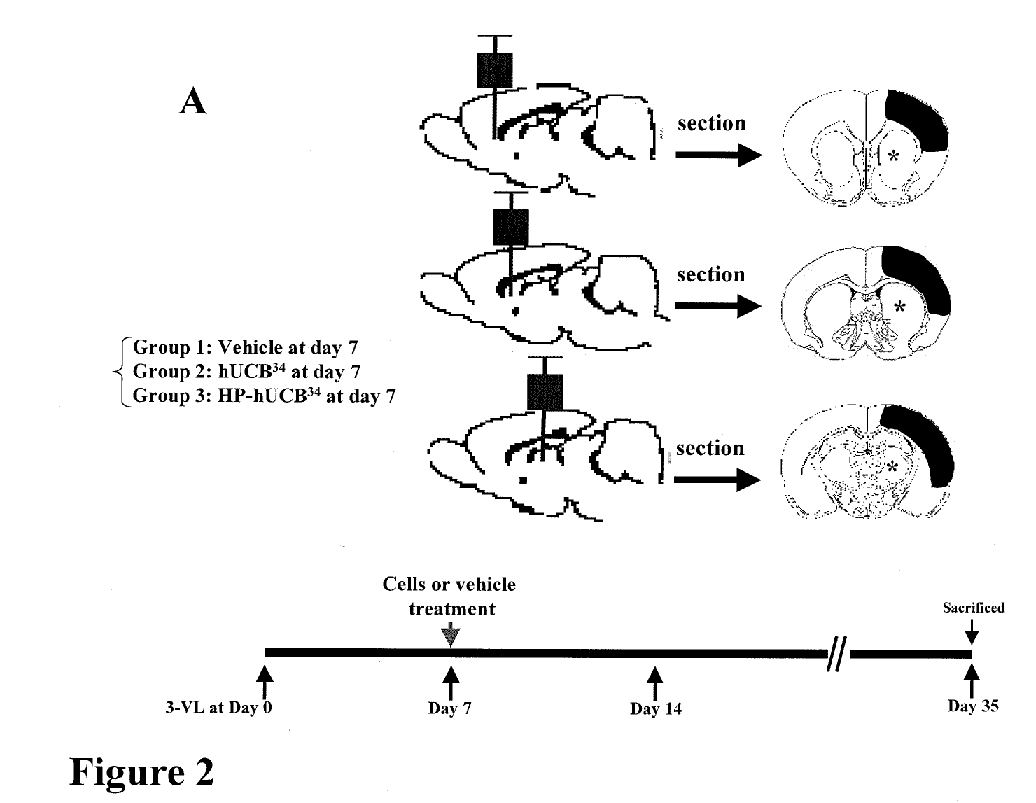

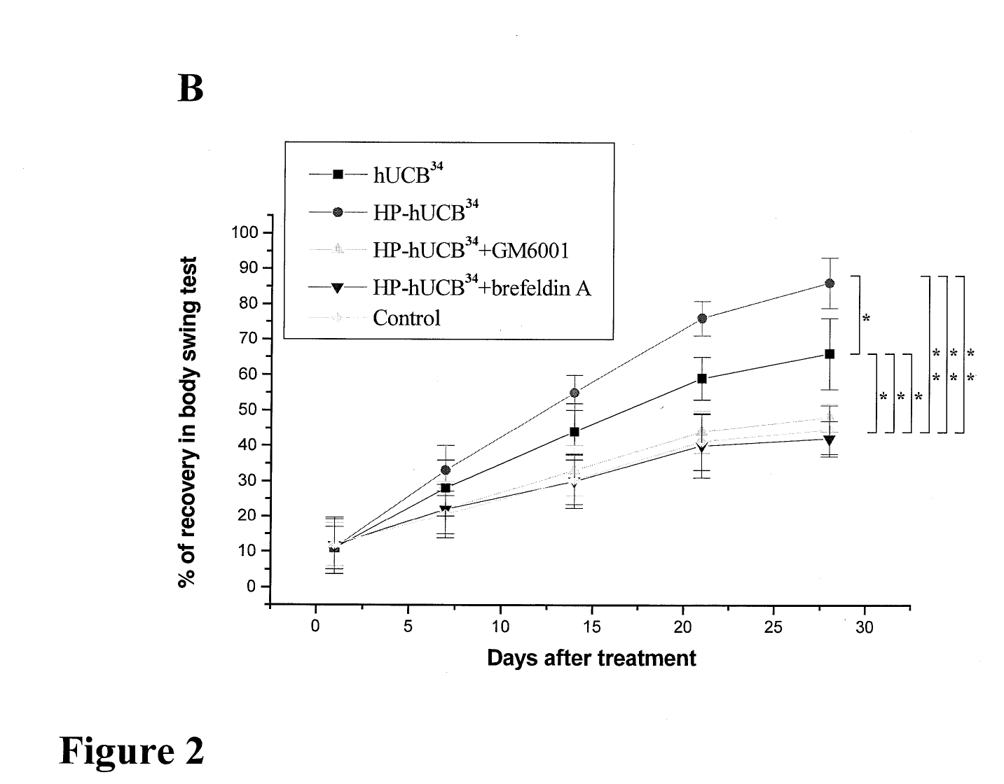

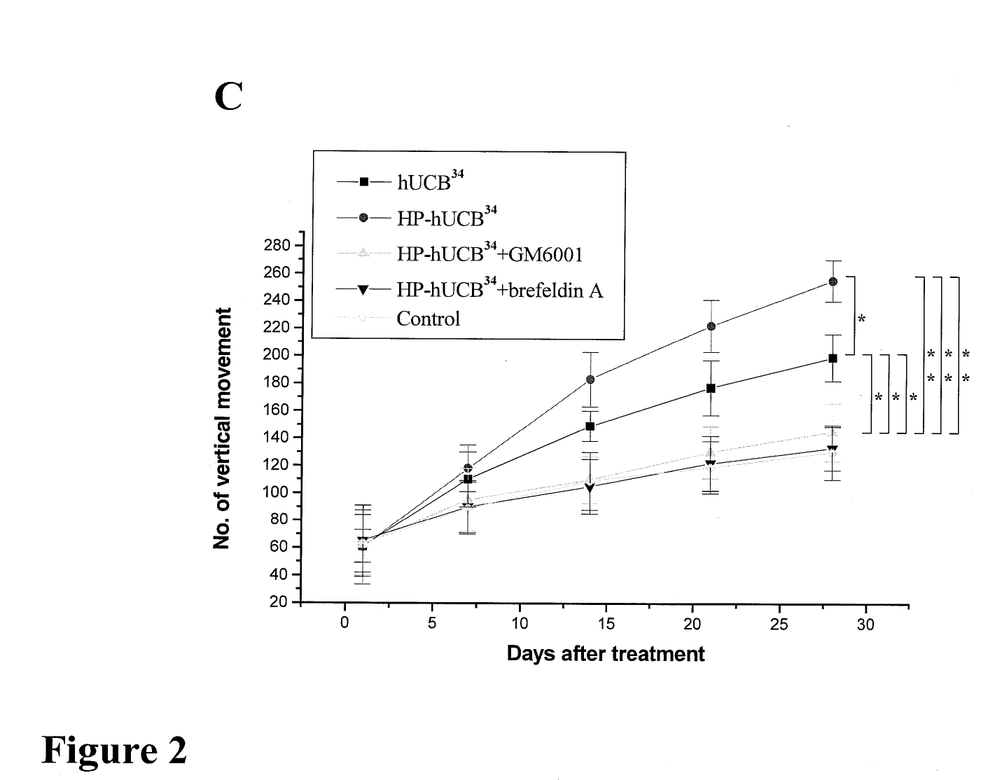

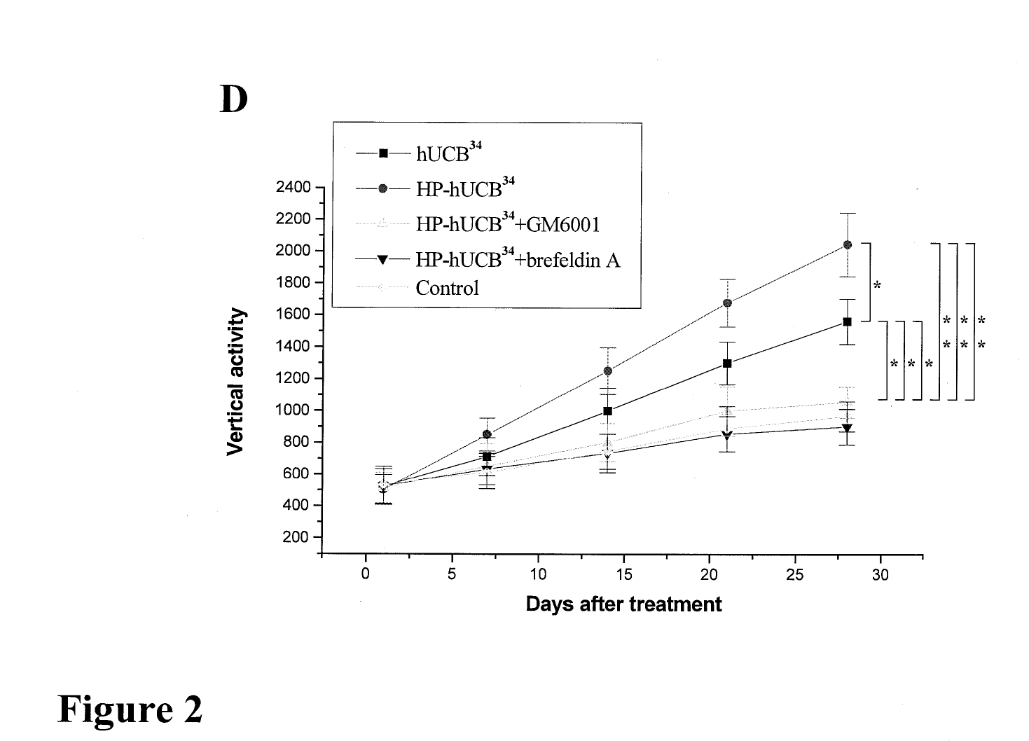

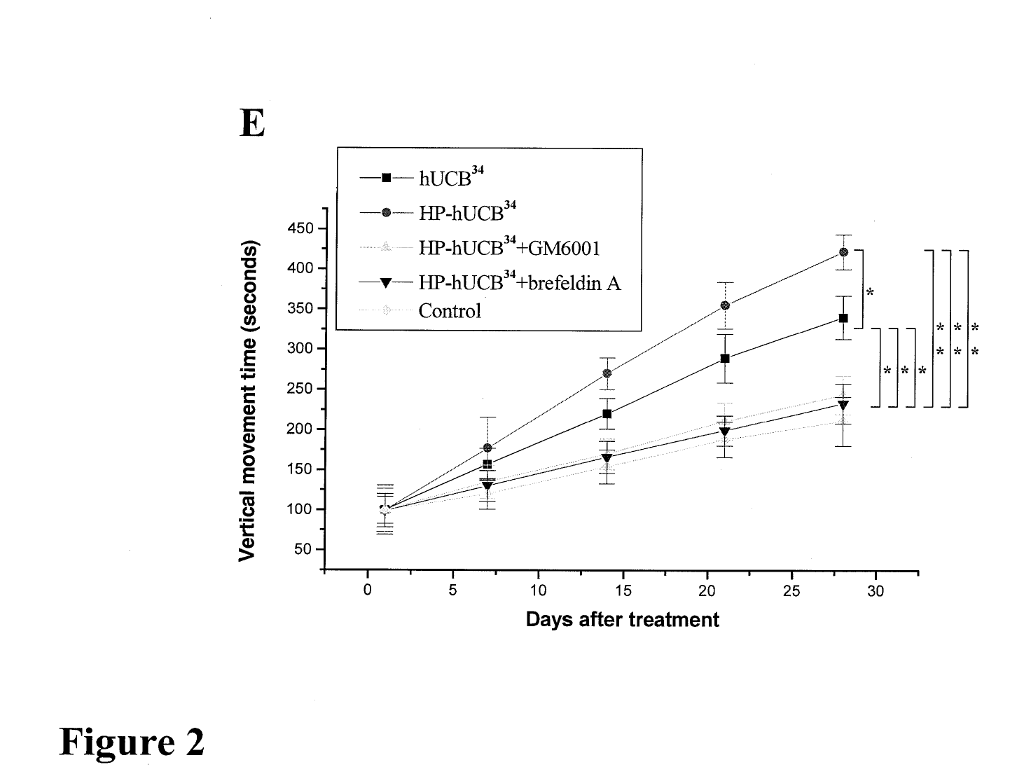

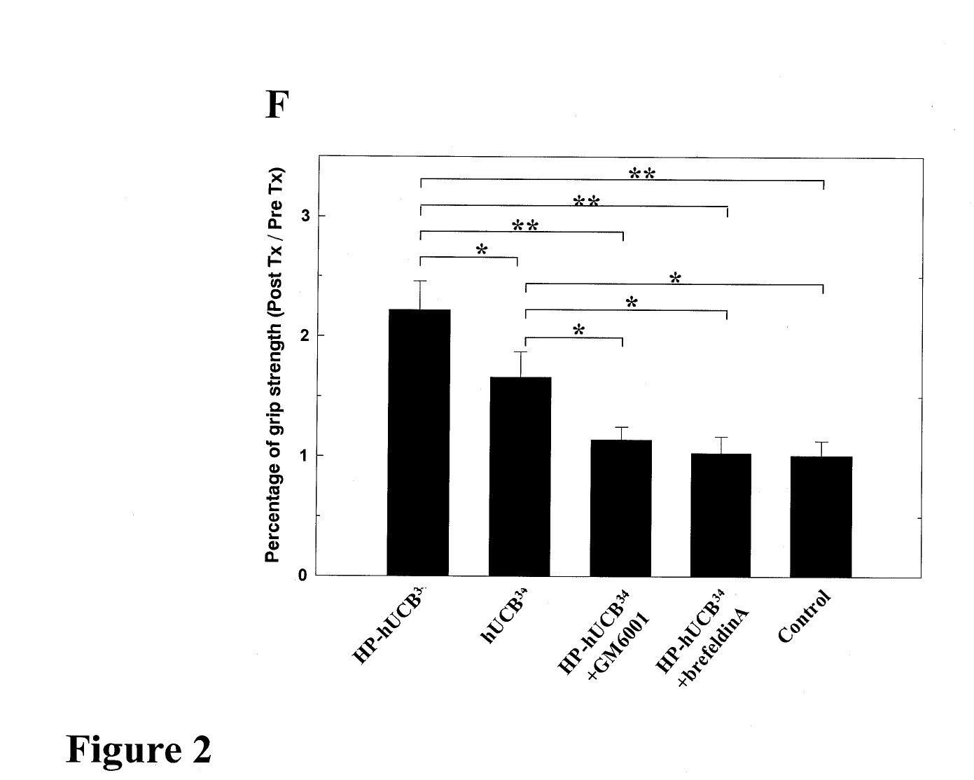

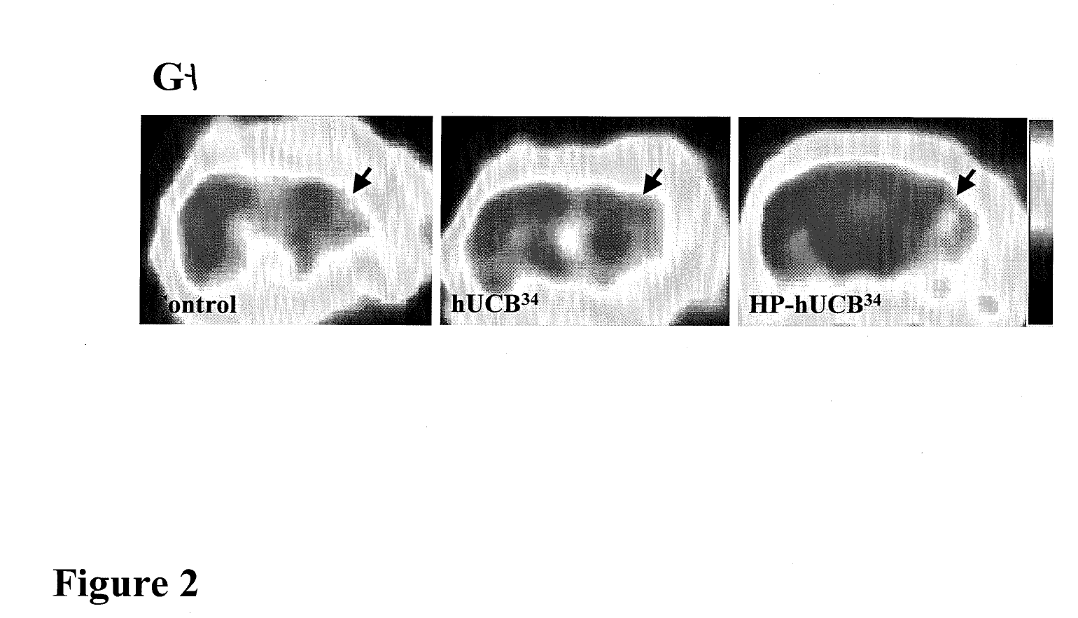

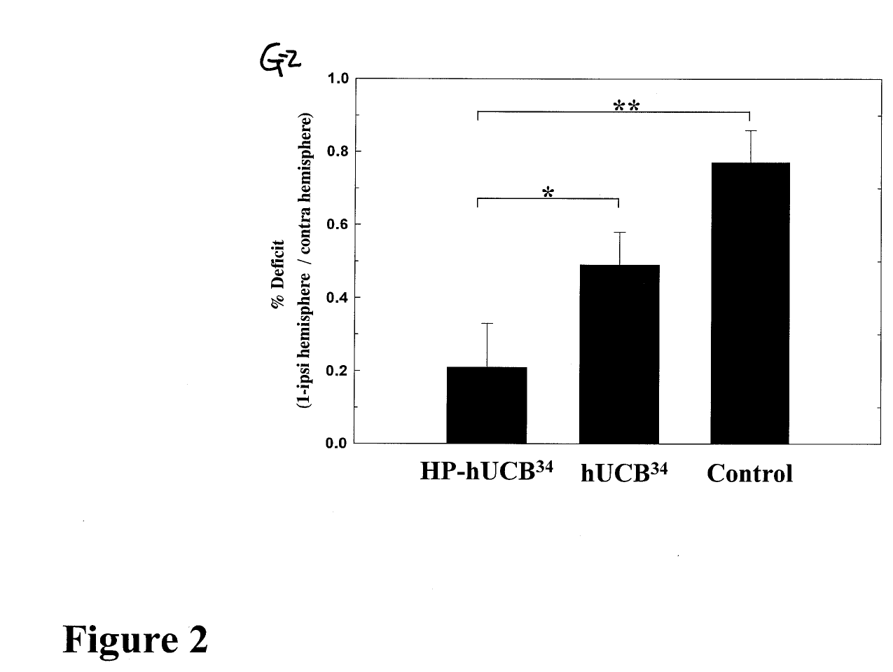

Experimental rats and mice were divided into three groups (FIG. 2A): Intracerebral transplantation of hUCB³⁴ with HP (HP-hUCB³⁴), intracerebral transplantation of hUCB³⁴ without HP (hUCB³⁴), and a vehicle-control group. All transplantation took place on day 7. The HP-hUCB³⁴ group was treated in 1% O₂ hypoxic conditions for 20-24 hours. Cerebral ischemia was induced in every experimental rat on day 0. At 7 days after cerebral ischemia, experimental rats in the two intracerebral hUCB³⁴ transplantation groups were injected stereotaxically with approximately 2×10⁵ cells of hUCB³⁴ labeled with bis-benzimide in a 3-5 μL PBS suspension through a 26- or 30-gauge Hamilton syringe into 3 cortical areas, 3.0 to 5.0 mm (2.0 to 3.0 mm for mice) below the dura. The approximate coordinates for these sites were 1.0 to 2.0 mm (0 to 1.0 mm for mice) anterior to the bregma and 3.5 to 4.0 mm (2.0 to 2.5 mm for mice) lateral to the midline, 0.5 to 1.5 mm (0 to 1.0 mm for mice) posterior to the bregma and 4.0 to 4.5 mm (2.0 to 3.5 mm for mice) lateral to the midline, and 3.0 to 4.0 mm (1.5 to 2.5 mm for mice) posterior to the bregma and 4.5 to 5.0 mm (2.0 to 3.0 mm for mice) lateral to the midline. The needle was retained in place for 5 minutes after each injection and a piece of bone wax was applied to the skull defects to prevent leakage of the injected solution. Rats in the vehicle-control group were treated with saline stereotaxically. Cyclosporin A (CsA, 10 mg/kg, ip, Novartis) injections were given daily to each experimental rat, and an equal volume of CsA or saline was injected to the transplantation groups and saline control group, respectively as previously described (Zhao et al., 2004, Cell Transplant 13:113-122). In order to inhibit the Epac1 activation in the transplanted HP-hUCBs³⁴, cells were incubated with 10 μg/mL brefeldin A (BFA, Sigma-Aldrich) for another 2-3 hours as described previously (Muller et al., Nature 410:50-56 and Aandahl et al., 2002, J Immunol 169:802-808). In addition, 100 mg/kg of a broad, class-specific metalloproteinase inhibitor (GM6001; Chemicon) was injected intra-peritoneally for 8 consecutive days as previously described (Lee et al., 2006, J Neurosci 26:3491-3495).

### Neurological Behavioral Measurements

Behavioral assessments were performed 3 days before cerebral ischemia. The tests measured: (a) body asymmetry as previously described, (b) locomotor activity as previously described, and (c) grip strength using a Grip Strength Meter (TSE-Systems, Germany) as previously described, with modification (Shyu et al., 2008, J Clin Invest 118:2482-2495).

### [¹⁸F]fluoro-2-deoxyglucose Positron Emission Tomography (FDG-PET) Examination

To assess the metabolic activity of brain tissue, experimental rats were examined using microPET scanning of [¹⁸F]fluoro-2-deoxyglucose (FDG) to measure relative metabolic activity as previously described (Carmichael et al., 2004, Stroke 35:758-763). In brief, ¹⁸F-FDG was synthesized as previously described (Hamacher et al., 1986, J Nucl Med 27:235-238) with an automated ¹⁸F-FDG synthesis system (Nihonkokan). Data were collected with a high-resolution small-animal PET (microPET Rodent R4, Concorde Microsystems Inc.). The system parameters were described by Visnyei et al. (Carmichael et al., 2004, Stroke 35:758-763). After one week of each treatment, the animals were anesthetized with chloral hydrate (0.4 g/kg, ip), fixed in a customized stereotactic head holder and positioned in the microPET scanner. The animals were then given an intravenous bolus injection of ¹⁸F-FDG (200-250 μCi/rat) dissolved in 0.5 mL of saline. Data acquisition began simultaneously with injections and continued for 60 min in one bed position using a 3-D acquisition protocol. The image data acquired from microPET were displayed and analyzed by IDL ver. 5.5 (Research Systems) and ASIPro ver. 3.2 (Concorde Microsystems) software. Coronal sections for striatal and cortical measurements represented brain areas between 0 and +1 mm from the bregma, while those for thalamic measurements represented areas between −2 and −3 mm from the bregma, as estimated by visual inspection of the unlesioned side. The relative metabolic activity in regions of interest (ROIs) of the striatum and cortex was expressed as a percentage deficit as previously described with modification (Carmichael et al., 2004, Stroke 35:758-763).

### Evaluation of hUCB³⁴ Transplantation Induced Angiogenesis

Cerebral microcirculation was analyzed by administering a fluorescent plasma marker (FITC-dextran, Sigma, USA) intravenously to rats and observing them with fluorescent microscopy (Carl Zeiss, Axiovert 200M, Germany), as previously described (Morris et al., 1999, Brain Res Brain Res Protoc 4:185-191). In addition, to quantify the cerebral blood vessel density, experimental rats were anesthetized with chloral hydrate and perfused with 4% paraformaldehyde. Histological sections (6 μm) were stained with specific antibody to CD-31 (1:100, BD-Pharmingen, USA), and conjugated with Cy-3 (1:500, Jackson Immunoresearch, PA, USA). The number of blood vessels was determined as previously described (Taguchi et al., 2004, J Clin Invest 114:330-338).

### Measurement of Cerebral Blood Flow (CBF)

Experimental rats were positioned in a stereotaxic frame and baseline local cortical blood flow (bCBF) was monitored after cerebral ischemia with a laser doppler flowmeter (LDF monitor, Moor Instrutments, Axminster, U.K.) in an anesthetized state (chloral hydrate) as previously described (Park et al., 2005, J Neurosci 25:1769-1777). In brief, CBF values were calculated as percentage increase compared to bCBF.

### In situ Zymography (ISZ) and Immunohistochemistry

In order to localize the gelatinase activity, in situ zymography was performed in brain section with gelatin labeled with FITC. Ischemic brains (at different time points: 3 days, 7 days, 14 days and 28 days after transplantation) were quickly removed without fixation and frozen on dry ice as previously described (Amantea et al., 2008, Neuroscience 152:8-17). After cryostat sectioning (20 μm per section), the specimens were incubated overnight at 37° C. in fluorescent-labeled gelatin (Invitrogen) according to the manufacturer's instructions. Using this technique, proteolytic digestion of the substrate results in unblocking of green fluorescence. ISZ was combined with immunohistochemistry for the neuron-specific marker of Neu-N and Epac1, the other alternative sections were subsequently fixed in 2% paraformaldehyde and subjected to double labeling using antibodies of Neu-N (1:200, Chemicon) and Epacl (1:400, Santa Cruz) conjugated with Cy3 (1:500; Jackson Immunoresearch) (Amantea et al., 2008, Neuroscience 152:8-17).

### Western Blot Assay

Protein expression in the right cortex and striatum region was also examined in the hUCB³⁴-treated and control animals using western blot analysis as described previously (Shyu et al., 2005, J Neurosci 25:8967-8977). In brief, experimental animals were decapitated at 3 days after cerebral ischemia. Samples of ischemic cerebral cortex were taken from the peripheral region of infarcted brains (penumbric region) and striatum. Western blot analysis was performed on these samples. Subsequently, ischemic brain tissue was homogenized and lysed in a buffer containing 320 mM sucrose, 5 mM HEPES, 1 μg/mL leupeptin, and 1 μg/mL aprotinin. Lysates were centrifuged at 13,000 g for 15 min. The resulting pellet was resuspended in sample buffer (62.5 mM Tris-HCl, 10% glycerol, 2% SDS, 0.1% bromophenol blue, and 50 mM DTT) and subjected to SDS-polyacrylamide gel (4-12%) electrophoresis. Then, the gel was transferred to a Hybond-P nylon membrane. This was followed by incubation with appropriately diluted antibodies of Bc1-2 (dilution 1:200; Santa Cruz, USA), Bc1-xL (dilution 1:200; Transduction Laboratories, USA), Bax (dilution 1:200; Santa Cruz, USA), Bad (dilution 1:200; Transduction Laboratories, USA), MMP2 (1:200, Abcam), Epac1 (1:400, Santa Cruz), CXCR4 (1:200, R&D System) and f3-Actin (dilution 1:2000, Santa Cruz, USA). Membrane blocking, primary and secondary antibody incubations, and chemiluminescence reactions were conducted for each antibody individually according to the manufacturer's protocol. The intensity of each band was measured using a Kodak Digital Science 1D Image Analysis System (Eastman Kodak, Rochester, N.Y.). The ratio of band intensity of Western blots in comparison with the internal control was calculated. Results were expressed as the mean value of the ratio±SEM for preparations.

### Gel Zymography (GZ)

Brain extract and cell lysate containing equal amounts of protein were loaded onto a 10% SDS-polyacrylamide gel containing gelatin (Bio-Rad, CA). After electrophoresis, gels were washed in 5% Triton X-100 and then incubated in MMP assay buffer (Bio-Rad). Bands were visualized with Coomassie Brilliant Blue and destained in 30% methanol and 10% acetic acid.

### Assessment of Neurite Regeneration In Vivo

Brain tissue samples were immunostained to measure neurite outgrowth.

Measurement of neurite regeneration was performed as described earlier (Cafferty et al., 2004, J Neurosci 24:4432-4443). Briefly, brain tissue samples from each experimental rat were fixed and immunostained with specific antibody against β-tubulin (1:400; Sigma). For quantification analysis, neurons with processes greater than twice the cell body diameter were counted as neurite-bearing cells. The length of the longest neurite of each neuron was measured from digitized images and quantified using imaging analysis software (SigmaScan 4.01.003).

### Transgenic and Knockout Mouse Lines

The Nestin-EGFP transgenic mice were a kind gift from Dr. Docherty (Bernardo et al., 2006, Mol Cell Endocrinol 253:14-21). The deficient mice of MMP9 (MMP9^{-/-}) were purchased from Jackson Laboratory (Bar Harbor, USA). MMP2 (MMP2^{-/-}) homozygous deficient mice were obtained by crossing heterozygotes from RIKEN Brain Science Institute. The Ethical Committee for animal research at China Medical University Hospital has reviewed and approved all animal experiments.

### Immunohistochemical Assessment of Brain Tissue

Animals were anesthetized with chloral hydrate (0.4 g/kg, ip) and their brains fixed by transcardial perfusion with saline, followed by perfusion with an immersion in 4% paraformaldehyde as previously described (Shyu et al., 2004, Circulation 110:1847-1854). The double immunofluorescence technique with specific antibodies against Epac1 (1:400, Santa Cruz), MMP2 (1:50, Abcam), GFAP (1:400; Sigma), MAP-2 (1:200; BM), Neu-N (1:200; Chemicon), and vWF (1:400; Sigma), conjugated with FITC (1:500; Jackson Immunoresearch) or Cy3 (1:500; Jackson Immunoresearch) has been described previously The tissue sections were analyzed with a Carl Zeiss LSM510 laser-scanning confocal microscope.

### Separation of GFP⁺ Neural Stem Cells (GFP⁺NSCs)

The brains of 3-day-old newborn transgenic Nestin-EGFP-057BL/6 mice were removed. After removal of the meninges, hippocampi and subventricular layers from the lateral wall of the lateral ventricle were aseptically isolated and dissociated as previously described (Wachs et al., 2003, Lab Invest 83:949-962). Then, cells were resuspended in Neurobasal (NB) medium (Gibco BRL, Germany) supplemented with B27 (Gibco BRL, Germany), 2 mM L-glutamine (PAN, Germany), 100 U/ml penicillin/0.1 mg/L streptomycin (Gibco, Germany). For maintenance and expansion of the cultures, the NB/B27 was further supplemented with 2 μg/mL heparin (Sigma, Germany), 20 ng/mL FGF-2 (R&D Systems, Germany) and 20 ng/mL EGF (R&D Systems, Germany). Cultures were maintained at 37° C. in a humidified incubator with 5% CO₂. GFP⁺NSC cultures from passage number 4 to 6 were used throughout this study.

### Co-culture of CD34+ Cells with GFP⁺NSCs

GFP⁺NSCs (1×10⁶) were harvested using trypsinization, and subdivided in each well of a 6-well tissue culture plate for co-culture with CD34⁺ cells. The immunoselected CD34⁺ cells ranging from 1×10⁴ cells/mL were resuspended in a 2 mL mixture containing 10% FBS, 2 mmol/L L-glutamine, 1× ITS-S (Life Technologies, San Francisco, Calif., USA), with saturating doses of recombinant human thrombopoietin (rhTPO; Kirin Brewery, Tokyo, Japan) at 50 ng/mL of a stem cell factor (SCF; Kirin Brewery, Tokyo, Japan), 50 ng/mL of a flt3-ligand (FL; R&D systems, Minneapolis, Minn., USA), 50 ng/mM interleukin-3 (IL-3; R&D systems, Minneapolis, Minn., USA), and 25 ng/mL interleukin-6 (IL-6; R&D systems, Minneapolis, Minn., USA), and DMEM in a 6-well tissue culture plate. One milliliter of fresh medium was added to each well every 2 days for a total of 8 days.

### Immunocytochemical Analysis

Cell cultures were washed with PBS and fixed for 30 minutes at room temperature in 4% paraformaldehyde as previously described (Cafferty et al., 2004, J Neurosci 24:4432-4443). After washing in PBS, the fixed cultured cells were treated for 30 minutes with blocking solution (10 g/L BSA, 0.03% Triton X-100, and 4% serum in PBS). Cells were incubated overnight at 4° C. with a primary antibody, including glial fibrillary acidic protein (GFAP, 1:300; Chemicon), stromal cell-derived factor 1 (SDF-1, 1:200; Chemokine), CXC receptor type 4 (CXCR4, 1:200; Chemokine), MMP9 (1:200, Abcam), βIII-tubulin (Tuj-1, 1:200; Chemicon), microtubular associated protein-2 (MAP-2, 1:300; Chemicon) and neuronal nuclear antigen (Neu-N, 1:50; Chemicon), for 3 hours followed by secondary antibody conjugated with FITC for 1 hour, and then rinsed 3 times in PBS. Finally, the slides were lightly counterstained with DAPI, washed with water, and then mounted.

### Assessment of Neurite Regeneration In Vitro

For β-tubulin immunostaining, cell cultures were washed with PBS and fixed for 30 minutes at room temperature in 4% paraformaldehyde. After washing in PBS, the fixed cultured cells were treated for 30 minutes with blocking solution (10 g/L BSA, 0.03% Triton X-100, and 4% serum in PBS). Cells were incubated overnight at 4° C. with an antibody against β-tubulin (1:200; Chemicon) for 3 hours followed by secondary antibody conjugated with FITC for 1 hour, and then rinsed 3 times in PBS. Finally, the slides were lightly counterstained with DAPI, washed with water, and then mounted. The number of neurite-bearing cells and neurite length were assessed as previously described with modification (Cafferty et al., 2004, J Neurosci 24:4432-4443). In brief, cells in each treated group were plated after OGD, fixed and immunostained for β-tubulin. For quantification, neurons with neurites were defined as those bearing a process greater than twice the length of the cell body. The length of the longest neurite of each neuron was measured from digitized images and quantified using the SigmaScan imaging analysis program (SigmaScan 4.01.003). All measurement data were calculated from triplicates of experiments.

### Statistical Analysis

All measurements in this study were performed blindly. Results are expressed as mean±SEM. The behavioral scores have been evaluated for normality. Student's t-tests were used to evaluate mean differences between the control and the treatment group. Data lacking normal distribution were analyzed by a one-way ANOVA. A value of P<0.05 was taken as significant.

### Results

### HP Enhances Preferential Effect on hUCB³⁴ Cells

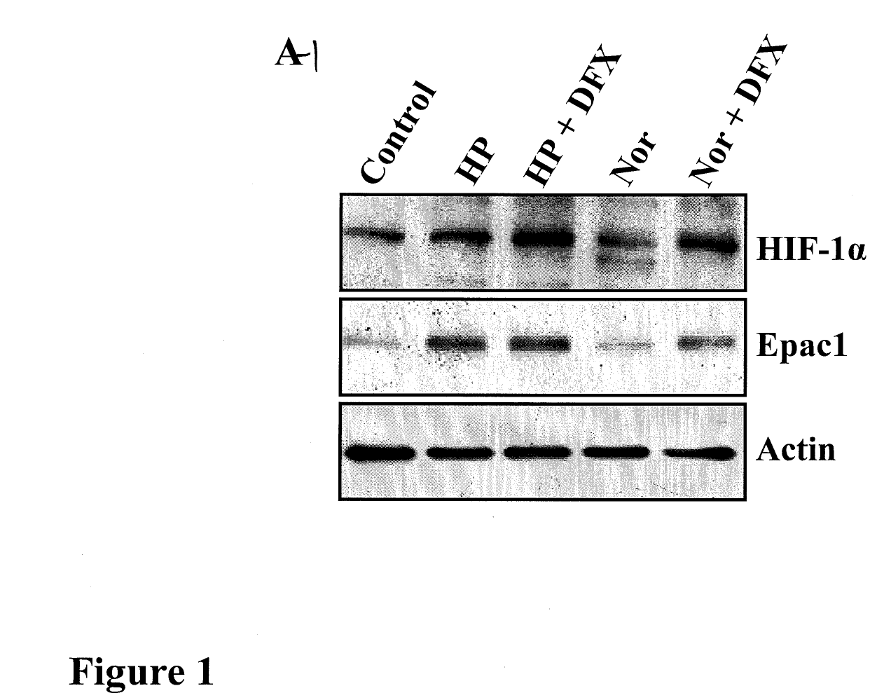

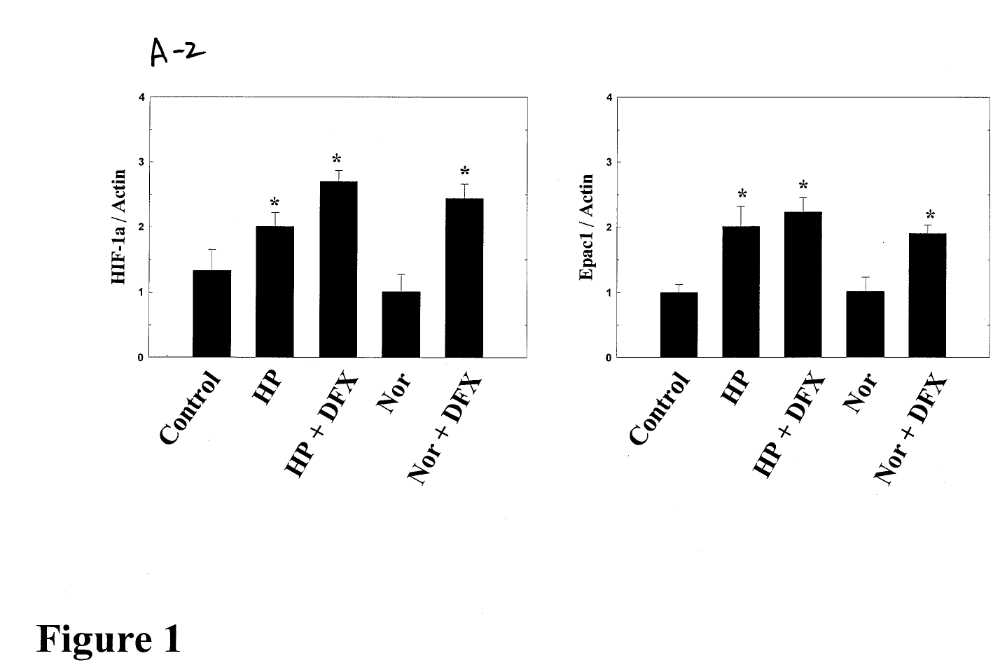

Western blot demonstrated that increased expression of Epacl in HP-hUCB³⁴ was confirmed at the protein level in hypoxic conditions for 20-24 h (FIGS. 1A-1 to 1A-2). HP-hUCB³⁴ treated with DFX, under either hypoxic or normoxic conditions, achieved much higher levels of Epac1 than control cells (FIG. 1A). We also detected an increase in the protein level of hypoxia-inducible factor-1α (HIF-1α) in HP- and DFX-treated hUCBs³⁴ (FIGS. 1A-1 to 1A-2). HP-hUCB³⁴ in short term hypoxia increased the active GTP-bound form of Rap1 reaching a maximum after 15 minutes of stimulation. These data demonstrate that HP-hUCB³⁴ expresses functional Epacl and that hypoxia is able to activate Rap1-GTP activity in HP-hUCB³⁴.

To obtain direct evidence for the interaction between HIF-1α and the Epac1 promoter, we used a Chromatin Immunoprecipitation (ChIP) assay to measure HIF-1α recruitment to the Epac1 promoter. Although no interaction between HIF-1α and the Epac1promoter was observed under normoxic conditions, recruitment of HIF-1α to the Epac1 promoter was clearly detected after 4 hours under hypoxic conditions.

### Intracerebral HP-hUCB³⁴ Transplantation Improves Neurological Behavior After Cerebral Ischemia

Neurological behavior measurement protocols were used to assess neurological function before and after MCA ligation in HP-hUCB³⁴-(n=10) and hUCB³⁴-treated rats (n=10), and control rats (n=10) (FIG. 2A). The behavioral measurement scores were all normalized to the baseline scores. Since cerebral ischemia causes imbalanced motor activity, all of the experimental rats developed significant body asymmetry, turning contralateral to the side of the ischemic brain on day 1 following cerebral ischemia. The hUCBs³⁴ were isolated by a magnetic bead separation method (MACS). The purity of isolated hUCB³⁴ was found to be greater than 90%, as established by FACS analysis (data not shown). From 14 to 28 days after treatment, rats treated with intracerebral HP-hUCB³⁴ transplantation, exhibited significantly reduced body asymmetry in comparison with hUCB³⁴-treated and control rats (FIG. 2B). Locomotor activity was examined before and after cerebral ischemia in all animals. Vertical activity, vertical movement time, and the number of vertical movements significantly increased between 14 and 28 days after cerebral ischemia in rats receiving HP-hUCB³⁴ transplantation in comparison with hUCB³⁴-treated and control rats (FIG. 2 C, D and E). Furthermore, measurement of grip strength was performed to examine the forelimb strength of all experimental rats before treatment and 28 days after each of the two treatments. The results revealed a higher ratio of grip strength in the HP-hUCB³⁴ group than in the hUCB³⁴-treated and control groups (FIG. 2F). In contrast, neurological behavior measurements from experimental rats receiving MMP inhibitor (GM6001) injection intraperitoneally after HP-hUCB³⁴ implantation (n=8) showed almost no recovery, the same as the measurements from the vehicle control rats after cerebral ischemia (FIG. 2 B-F). In addition, the degree of neurological dysfunction improvement after brefeldin A-incubated HP-hUCB³⁴ implantation in ischemic rats could be down-regulated to the same extent as that of control group by inhibiting the Epacl activation (n=8) (FIG. 2 B-F).

### Glucose Metabolic Activity is Enhanced in HP-hUCB³⁴-treated Stroke Rats

To verify whether intracerebral HP-hUCB³⁴ implantation could enhance glucose metabolic activity, each experimental rat was examined by ¹⁸FDG-PET. Glucose metabolism was measured by FDG microPET one week after each treatment. The uptake of FDG on the microPET image showed a striking increase in FDG uptake over the right cortex of the HP-hUCB³⁴-treated group (FIGS. 2G-1 to 2G-2). Semiquantitative measurement of relative glucose metabolic activity in the right hemisphere (relative to the non-stroke hemisphere) revealed significant enhancement in the HP-hUCB³⁴-treated rats (n=8) compared to the hUCB³⁴-treated (n=8) and control rats (n=8) (FIG. 2G-2).

### Intracerebral HP-hUCB³⁴ Transplantation Enhances Cells Engraftment and Neural Differentiation In Vivo

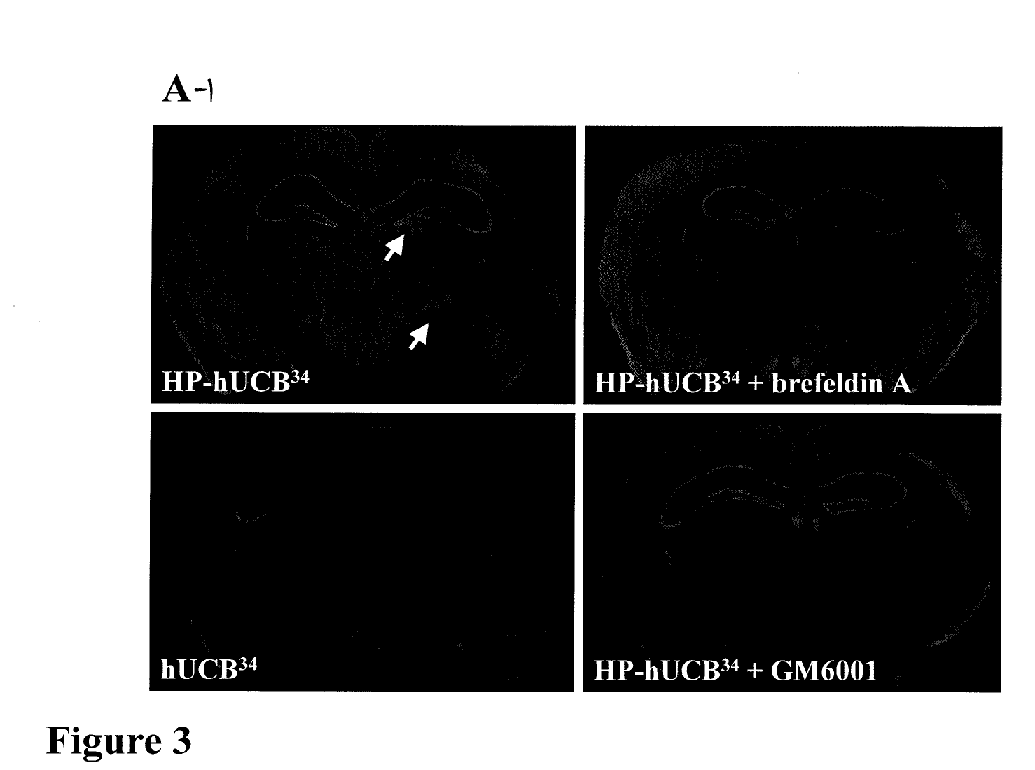

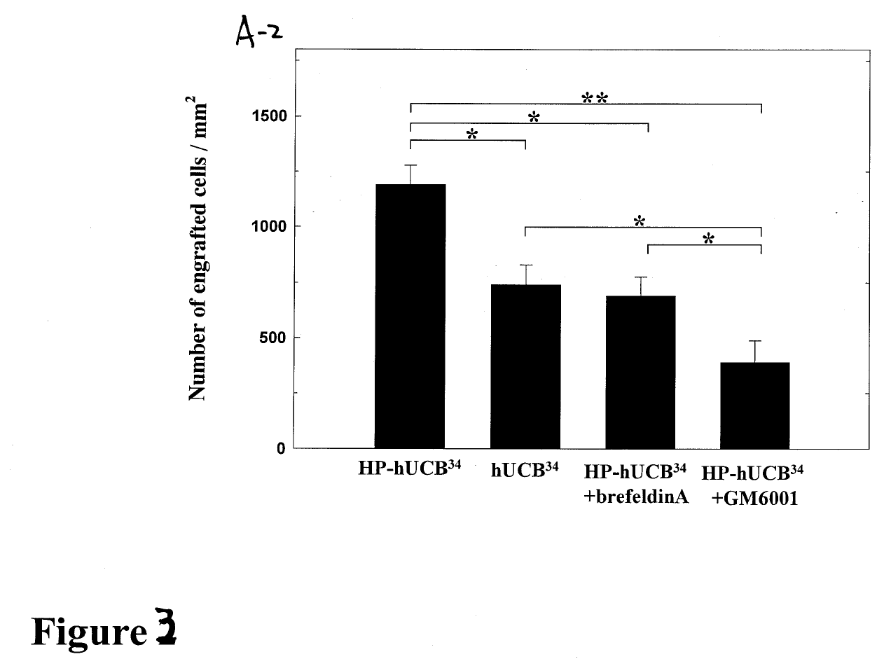

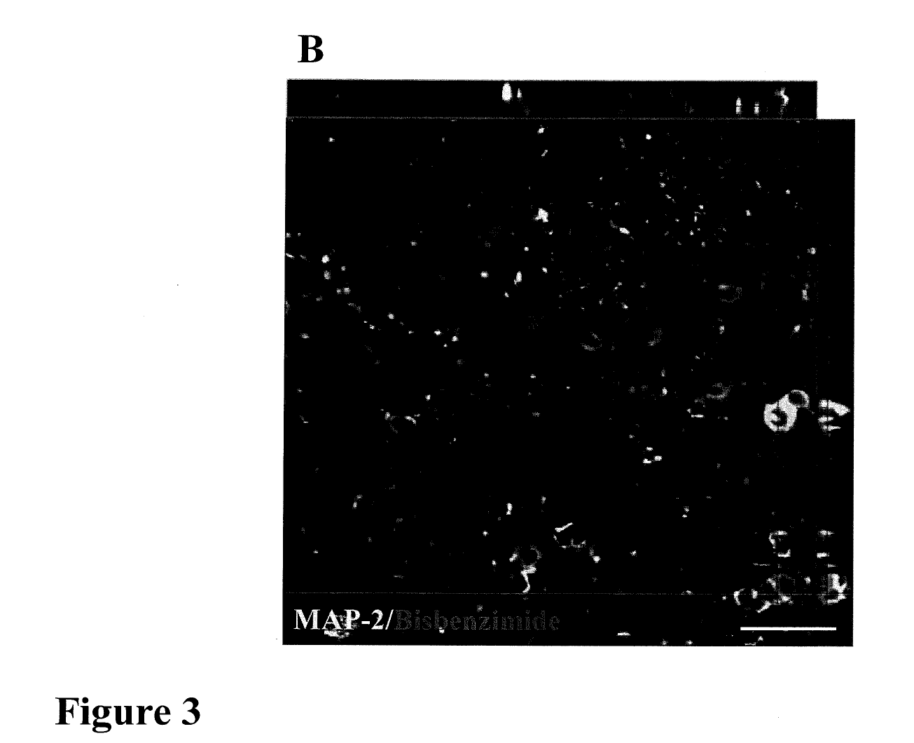

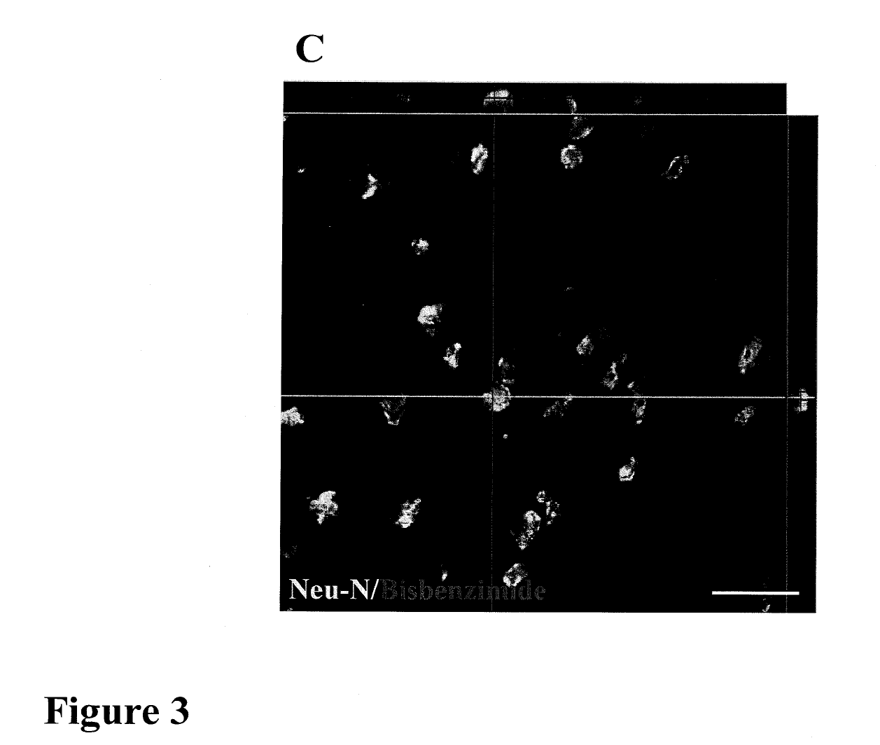

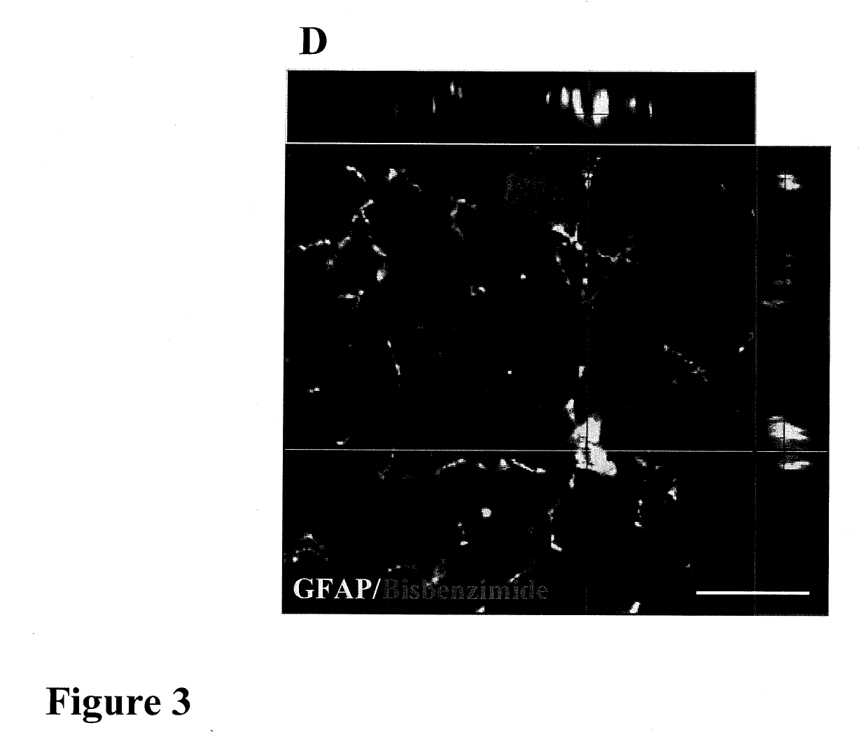

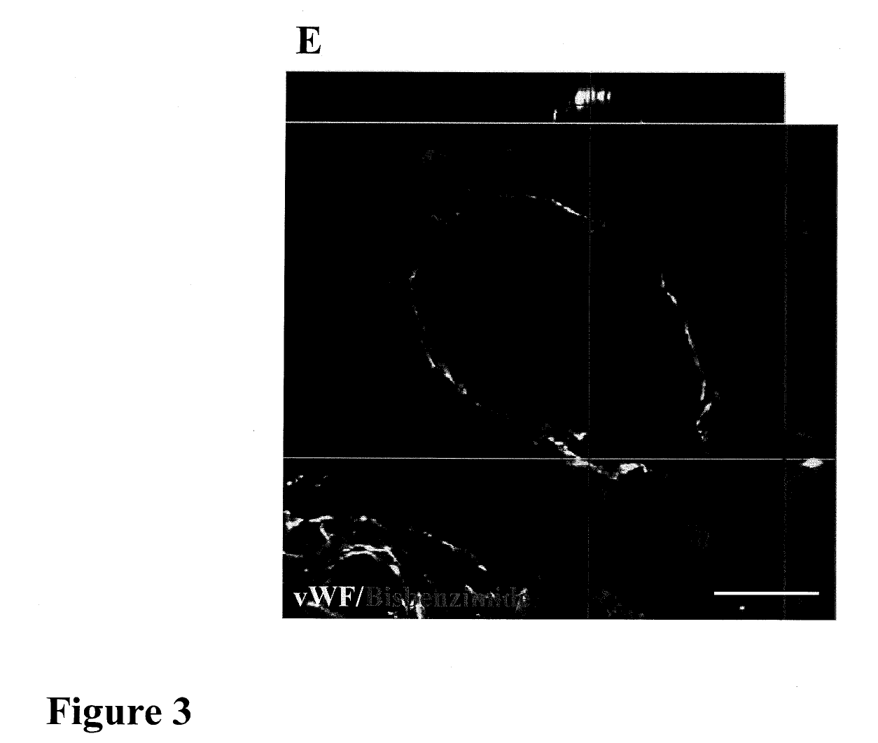

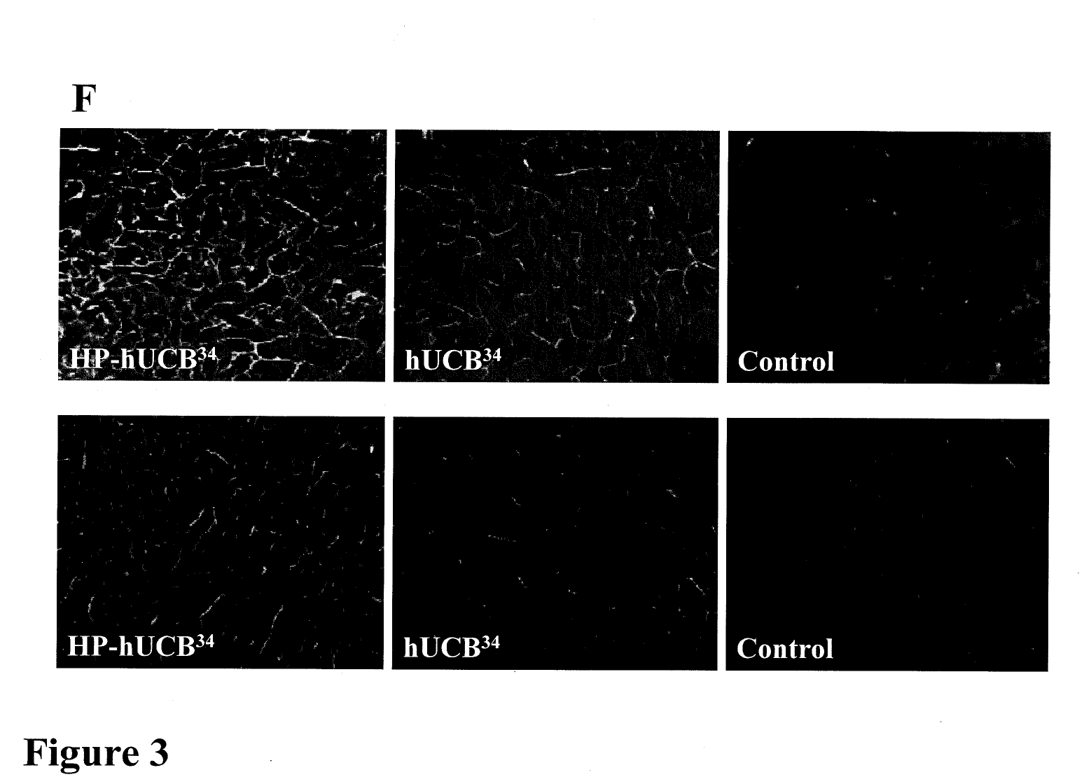

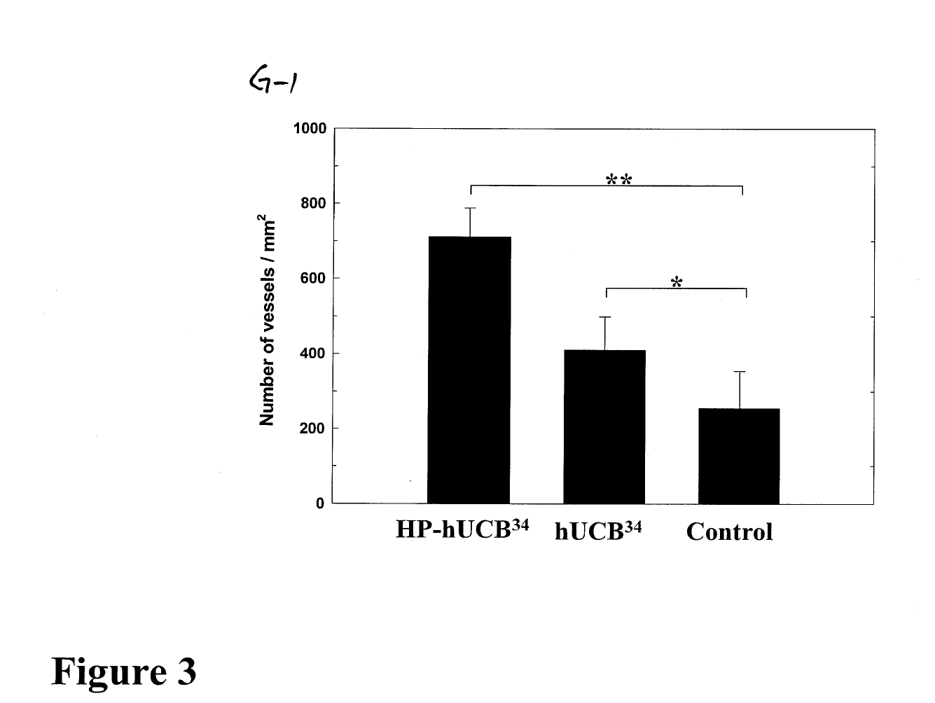

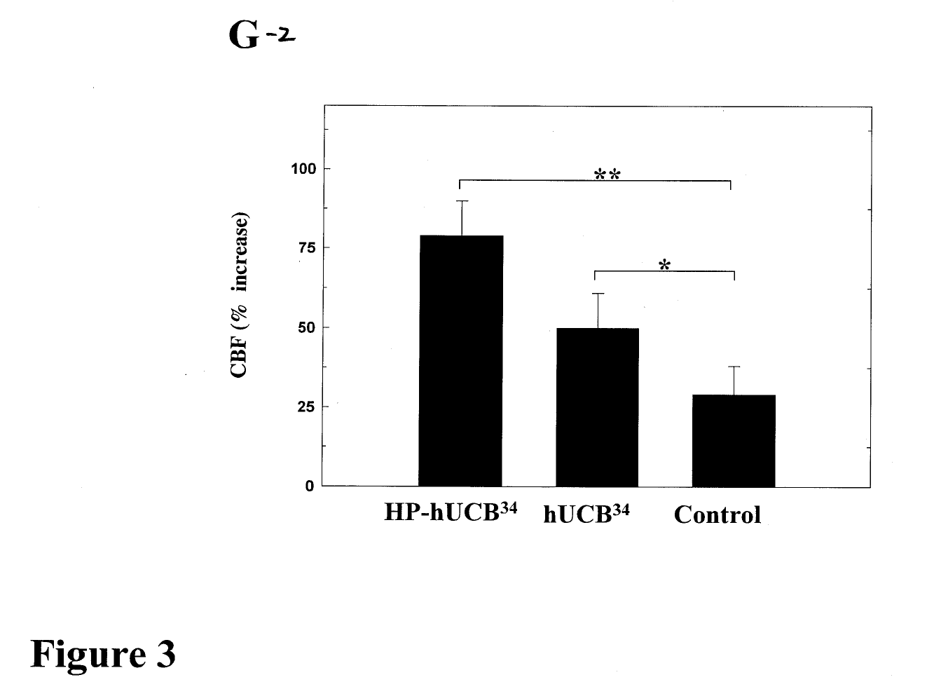

To determine whether exogenously transplanted HP-hUCB³⁴ could engraft into the ischemic brain and differentiate into neurons, and glial cells in the ischemic brains of experimental rats, immunoflourescent colocalization studies using a Laser-Scaning Confocal Microscope were performed. Implanted HP-hUCBs³⁴ labeled with bisbenzimide were well engrafted in the ischemic brain (FIGS. 3A-1 and 3A-2). A colocalization study showed that some bis-benzimide labeled cells colocalized with antibodies for MAP-2, Neu-N, and GFAP (FIGS. 3B-D) in the penumbra of HP-hUCB³⁴-treated ischemic rat brains. Differentiation rate in the HP-hUCB³⁴-treated rats (≈5.5% MAP-2⁺, ≈4% Neu-N⁺ and ≈9.5% GFAP⁺) (n=8) was also higher than that of hUCB³⁴-treated rats (≈3% MAP-2⁺, ≈2% Neu-N⁺ and ≈5% GFAP⁺) (n=8). In addition, the number of engrafted cells in the HP-hUCB³⁴-treated rats was also diminished by the injection of GM6001 (n=8) and brefeldin A-pretreatment (n=8) (FIG. 3A-2).

### Intracerebral HP-hUCB³⁴ Transplantation Induces Angiogenesis to Facilitate Cerebral Blood Flow (rCBF)

To determine whether HP-hUCB³⁴ could induce angiogenesis, double immunofluorescent staining, FITC-dextran perfusion studies, and blood vessel density assays were performed on brain slices from HP-hUCB³⁴-treated, hUCB³⁴-treated and vehicle-control treated rats. The results indicated that several implanted-HP-hUCB³⁴ (bisbenzimide-labeled) showed vascular phenotypes (vWF cells) around the perivascular and endothelial regions (FIG. 3E) in the penumbric region of ischemic brain. Visual inspection indicated that treatment with HP-hUCB³⁴ (n=8) significantly enhanced cerebral microvascular perfusion with FITC-dextran in comparison with hUCB³⁴ treatment (n=8) and control (n=8) (FIG. 3F). Quantitative measurement of blood vessel density examined by immunostaining of CD31 (FIG. 3F) revealed that ischemic rats treated with HP-hUCB³⁴ (n=6) showed significantly more neovasculature in the penumbric region than ischemic rats treated with hUCB³⁴ (n=8) or control rats (n=8) (FIG. 3F).

In order to verify whether increased blood vessel density could enhance functional CBF in the ischemic brain, experimental rats were monitored by laser doppler flowmetry (LDF) under anesthesia after cerebral ischemia. At one week after cerebral ischemia, there was a significant increase in CBF in the middle cerebral artery cortex of the HP-hUCB³⁴-treated rats (n=8) compared with hUCB³⁴-treated (n=8) and control rats (n=8) (FIGS. 3G-1 and 3G-2).

### Intracerebral HP-hUCB³⁴ Transplantation Rescues Neural Tissue by Increasing the Expression of Anti-Apoptotic Protein, Epac1, and MMP2

To investigate the molecular mechanism underlying the plastic effect of HP-hUCB³⁴ implantation, we examined the expression of apoptosis-related proteins. Western blot showed significantly upregulated expression of antiapoptotic proteins such as Bc1-2 in HP-hUCB³⁴-treated rats (n=6) at 3 days after implantation compared with hUCB³⁴-treated (n=6) and control rats (n=6) (FIGS. 4A-1 and 4A-2).

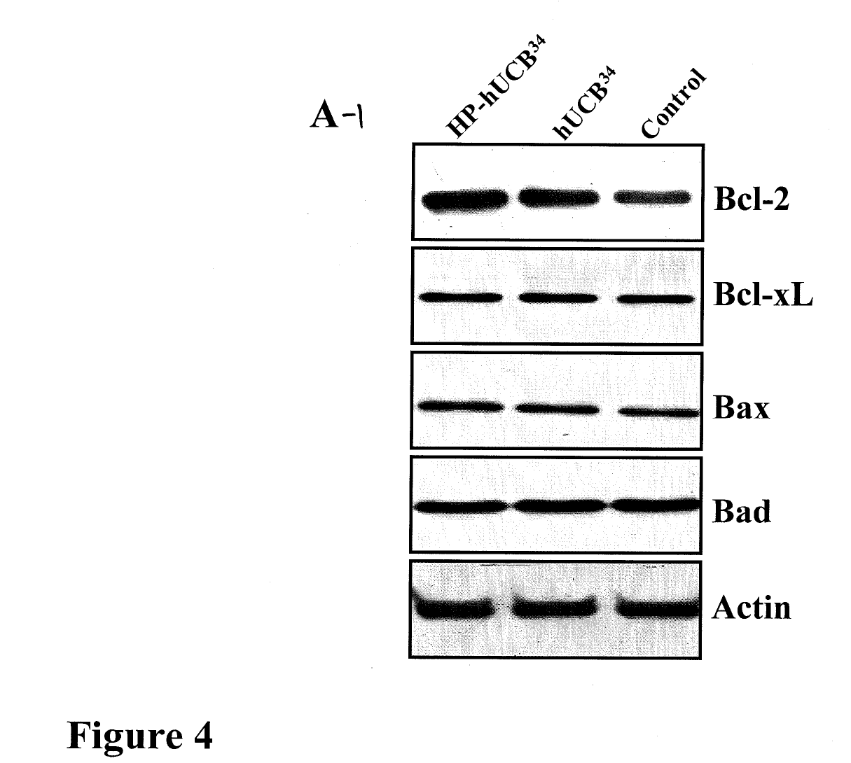

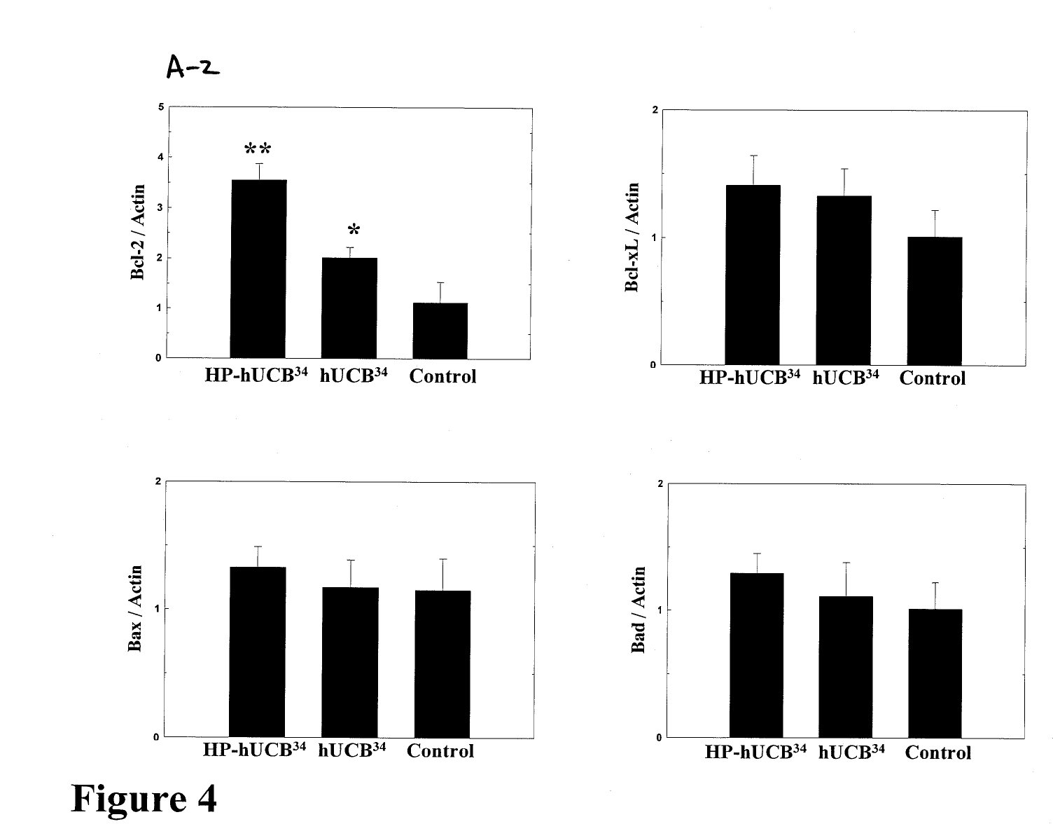

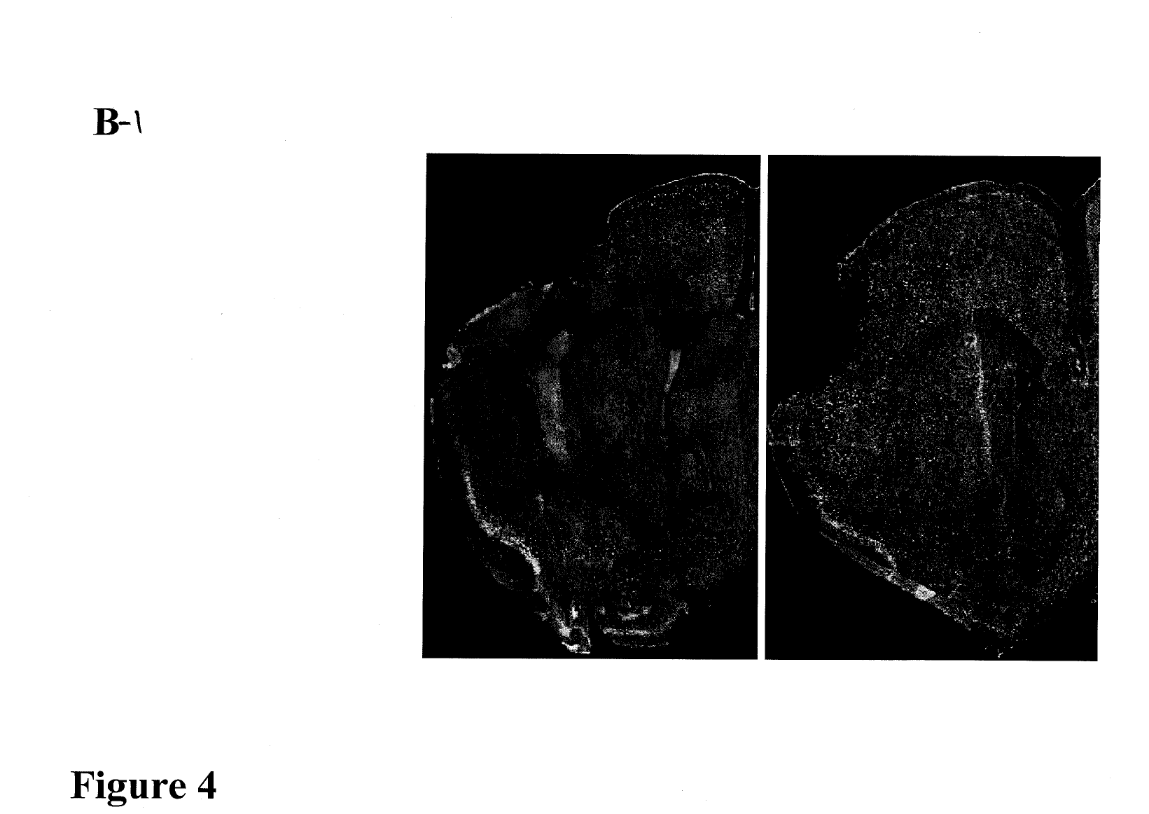

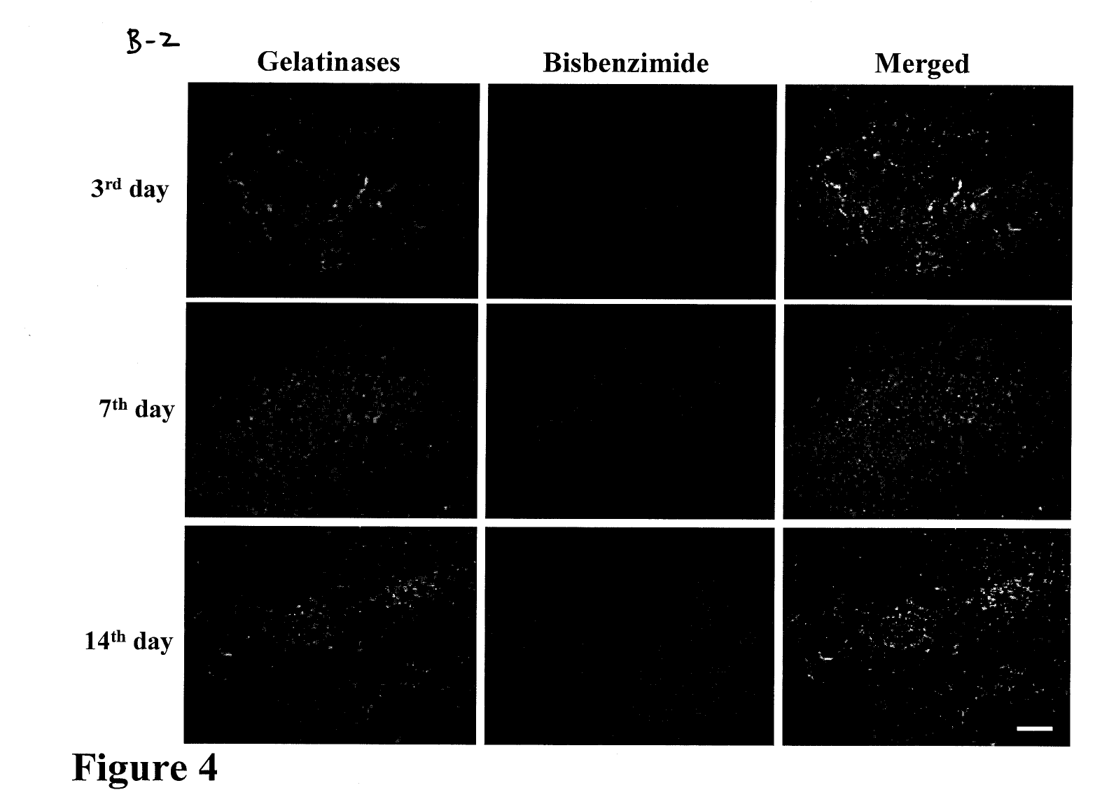

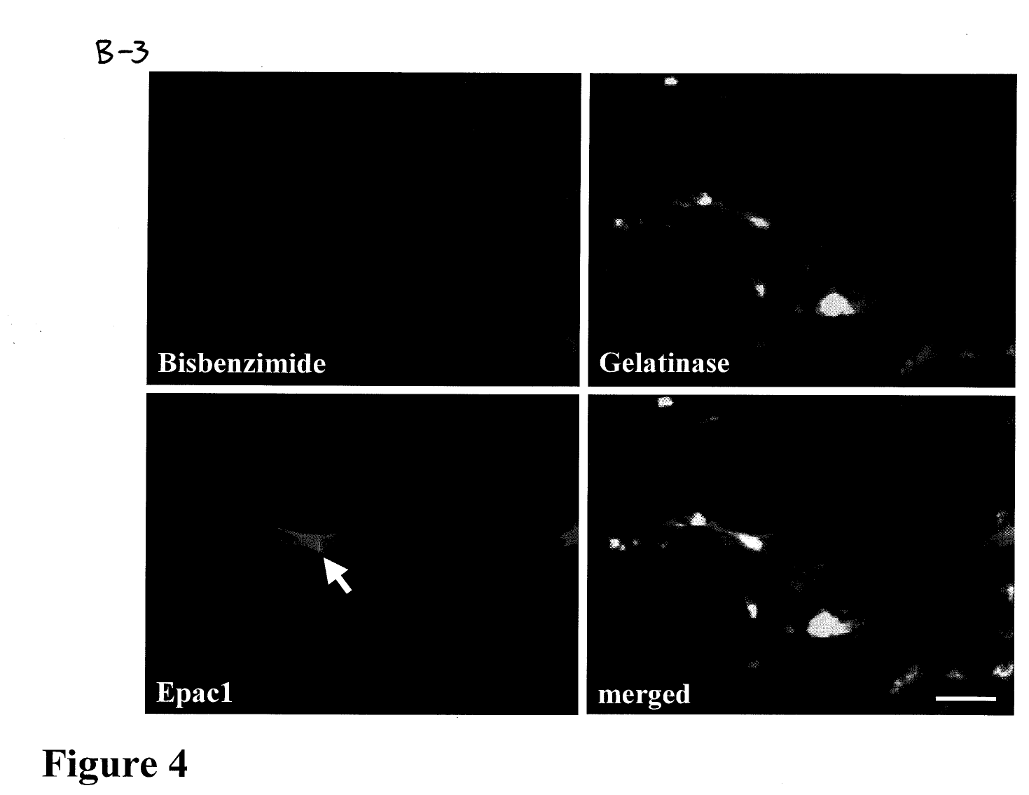

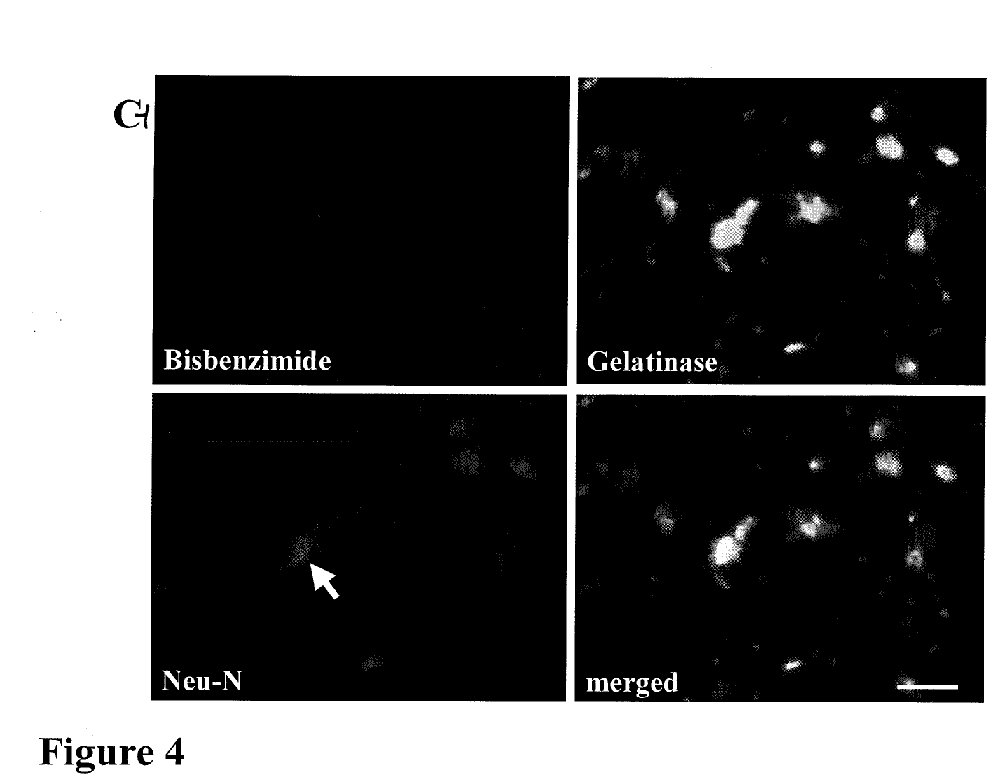

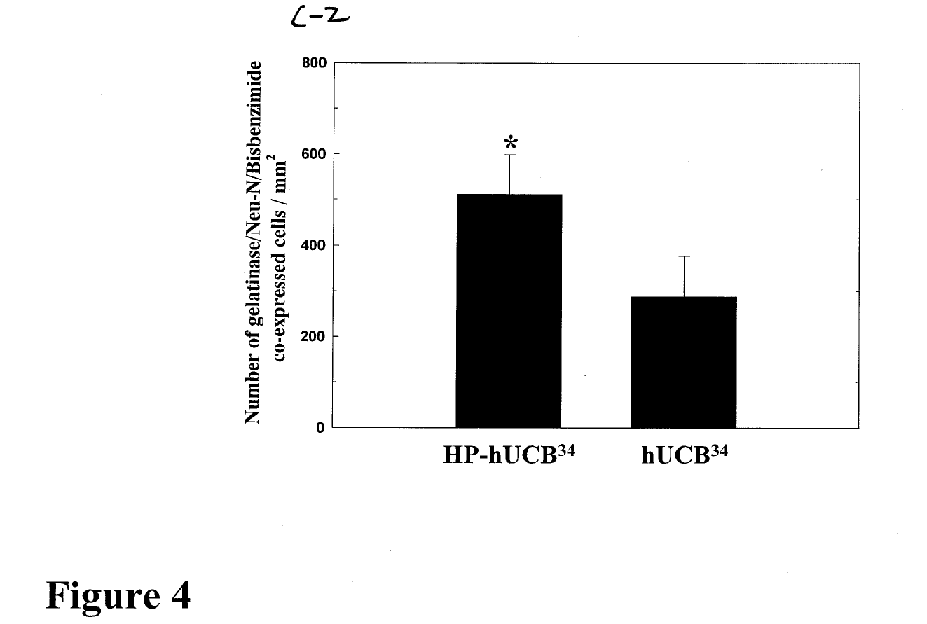

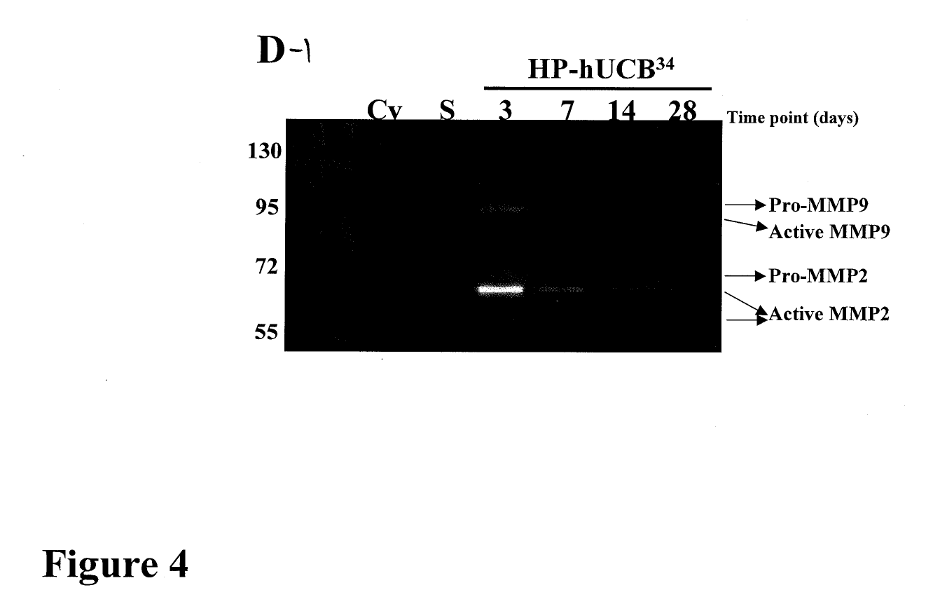

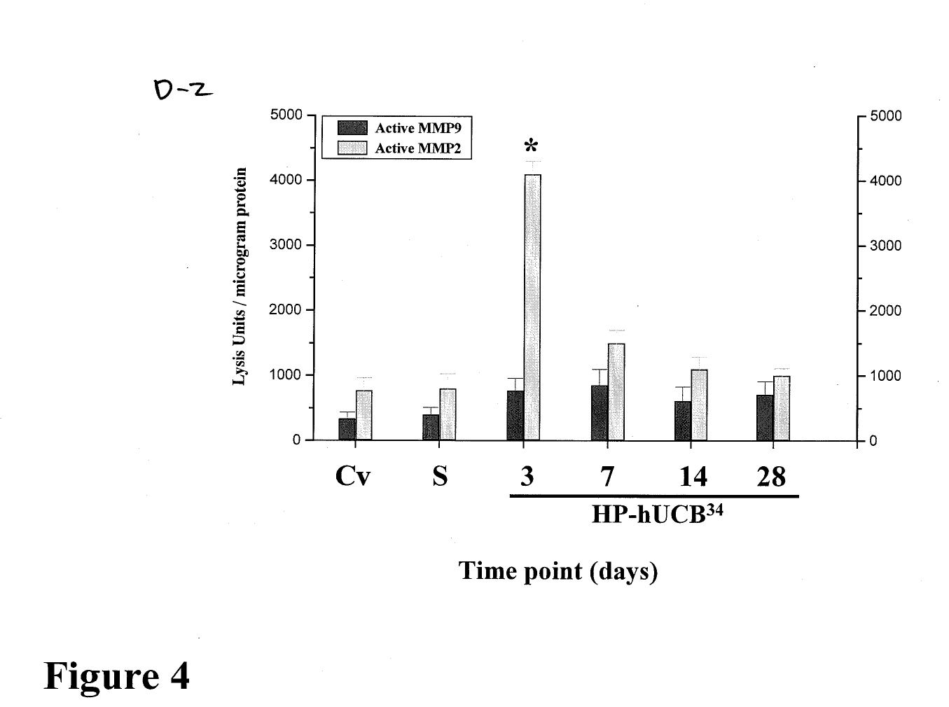

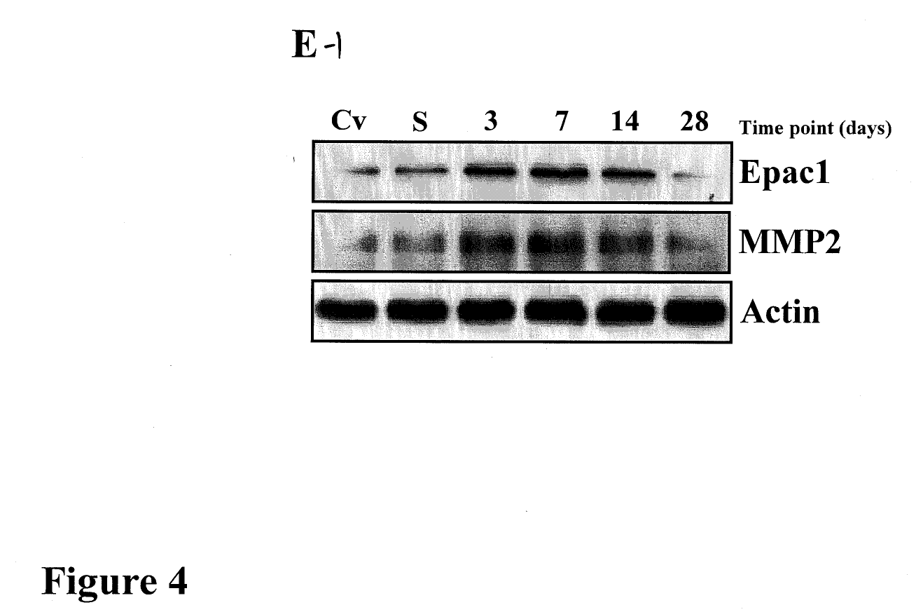

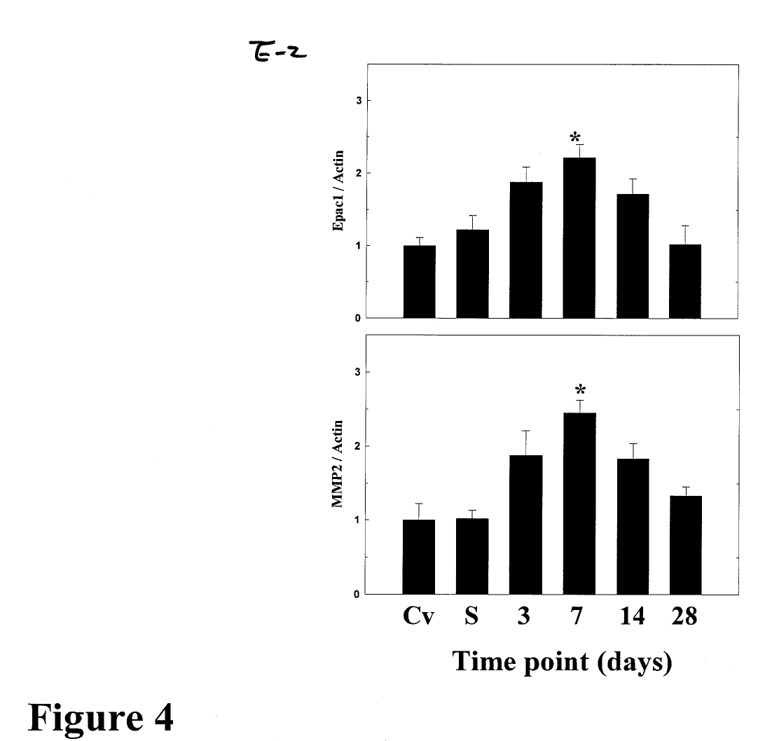

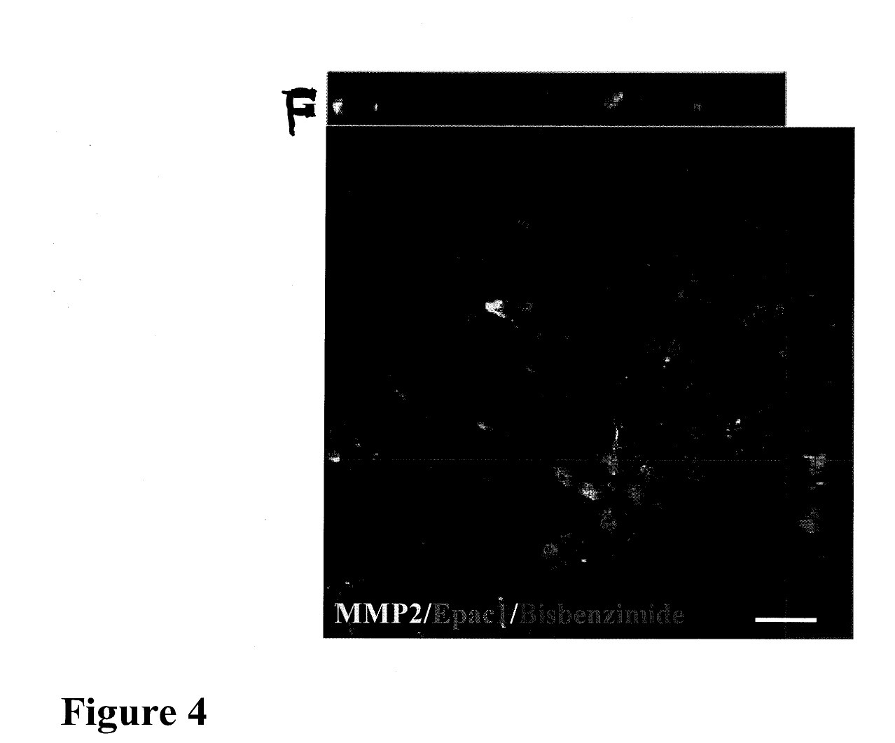

To examine whether HP-hUCB³⁴ implantation upregulates Epac1, and MMP2 expression in cerebral ischemic rats, we used in situ zymography (ISZ), gel zymography (GZ), immunohistochemistry (IHC) and western blot analysis. ISZ demonstrated that active gelatinase was present throughout the whole brain, especially over peri-implanted region after HP-hUCB³⁴ transplantation (FIGS. 4B-1 to 4B-3). Increased genatinase activity in ISZ showed coexpressed with Epacl after HP-hUCB³⁴ implantation (FIGS. 4B-2 and 4B-3). At 7 days after implantation, ISZ combined with IHC analysis showed that there were more gelatinase-Neu-N-bisbenzimide coexpressed cells in the brain sections of HP-hUCB³⁴-treated rats (n=6), than in hUCB³⁴-treated rats (n=6) (FIGS. 4C-1 and 4C-2). GZ showed that gelatinase activity (MMP2) was significantly increased in HP-hUCB³⁴-treated rats (n=6) compared to hUCB³⁴-treated rats (n=6) at 7 days after implantation (FIGS. 4D-1 and 4D-2). Western blot protein expression profiles showed a significant enhancement of Epac1, and MMP2 after 3 to 7 days in HP-hUCB³⁴ implanted rats (n=6) compared to hUCB³⁴-treated (S) (n=6) or vehicle-control rats (Cv) (n=6) (FIGS. 4E-1 and 4E-2). Transplantation of HP-hUCB³⁴ increased the active GTP-bound form of Rap1 reaching a maximum after 3 days of implantation. In IHC study, 3D colocalization showed that HP-hUCB³⁴ implantation enhanced the co-expression of Epac1, and MMP2 in the engrafted cells (FIG. 4F) at 7 days after treatment.

### Intracerebral HP-hUCB³⁴ Transplantation Enhances Neurogenesis to Promote Neurite Regeneration In Vivo

To determine whether transplanted HP-hUCB³⁴ could differentiate into neurons and glial cells modulated by Epac1 activation in ischemic brains of the experimental rats, immunoflourescent colocalization studies using Laser-Scaning Confocal Microscope were performed. In three dimensional colocalization study, the results showed that some bis-benzimide labeled cells colocalized with Epac1⁺ cells, and further with either MAP-2⁺, Neu-N⁺ or GFAP⁺ cells (FIGS. 5A-1 to 5A-3) in the penumbra of HP-hUCB³⁴- and hUCB³⁴-treated ischemic rat brains.

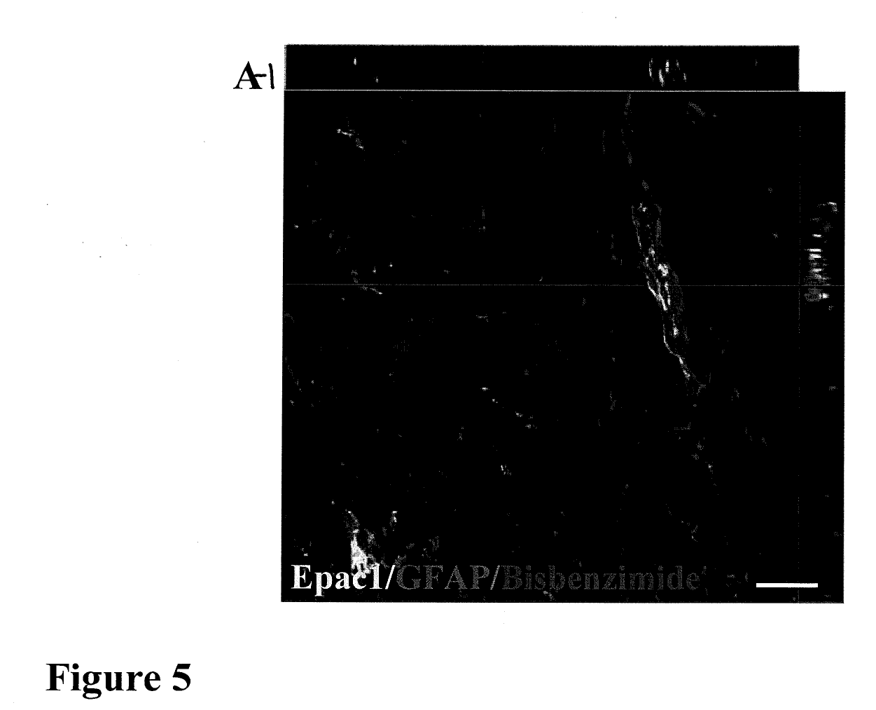

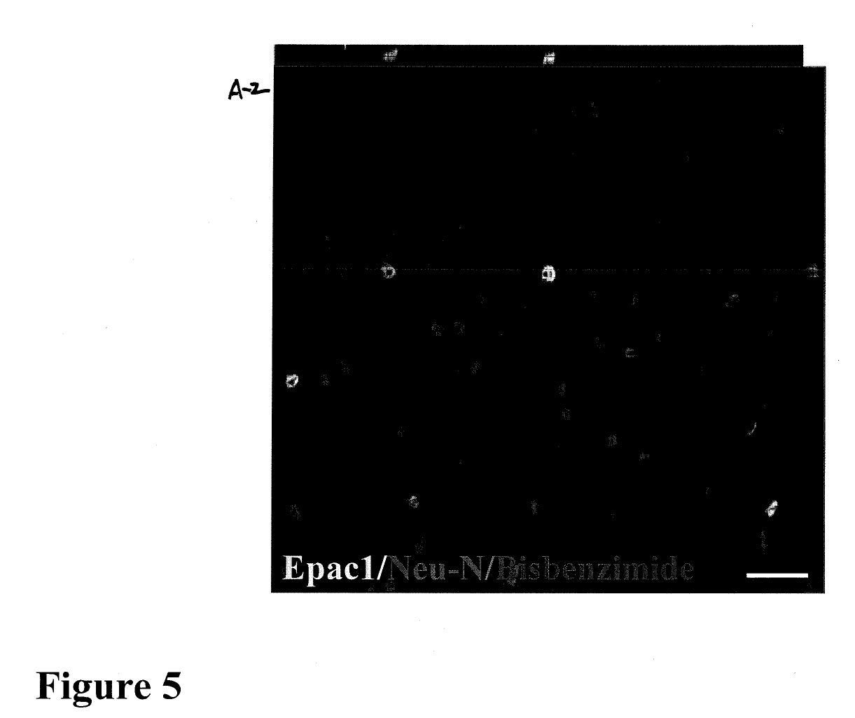

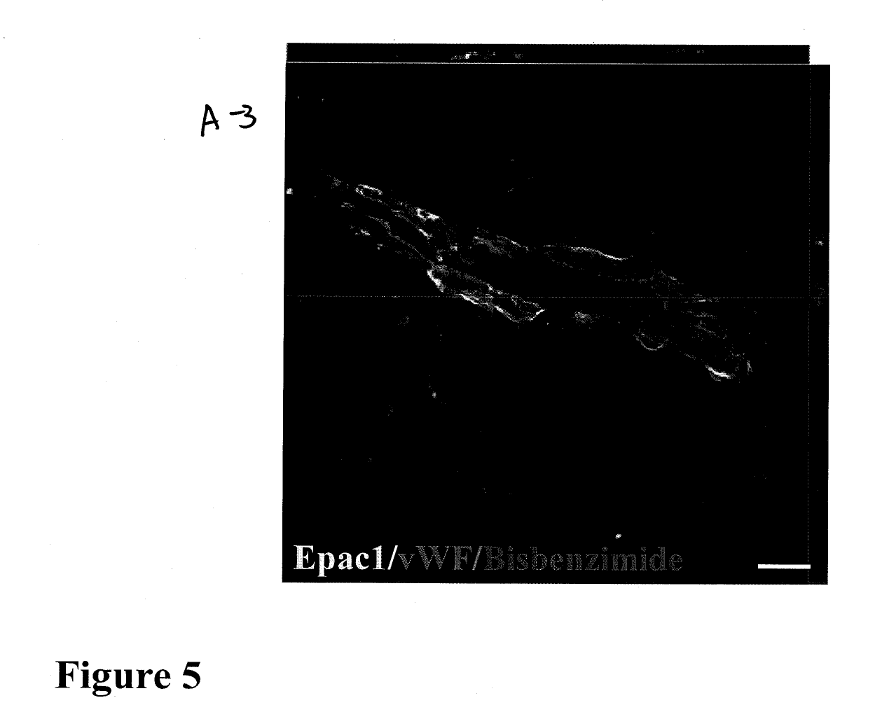

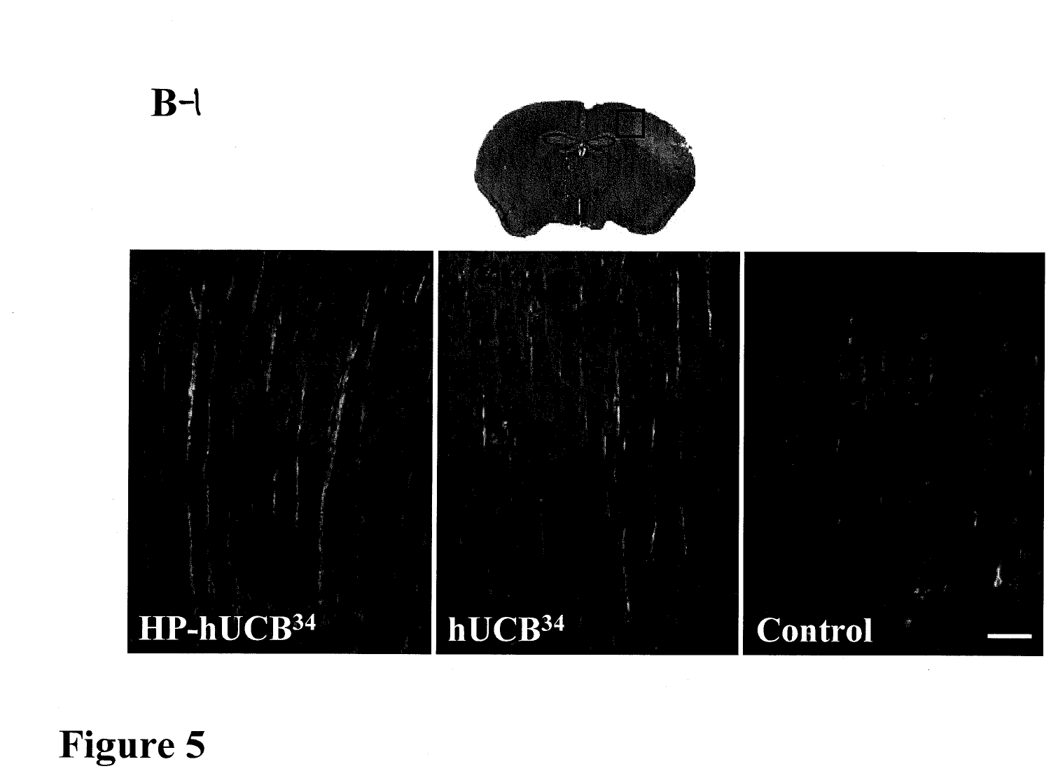

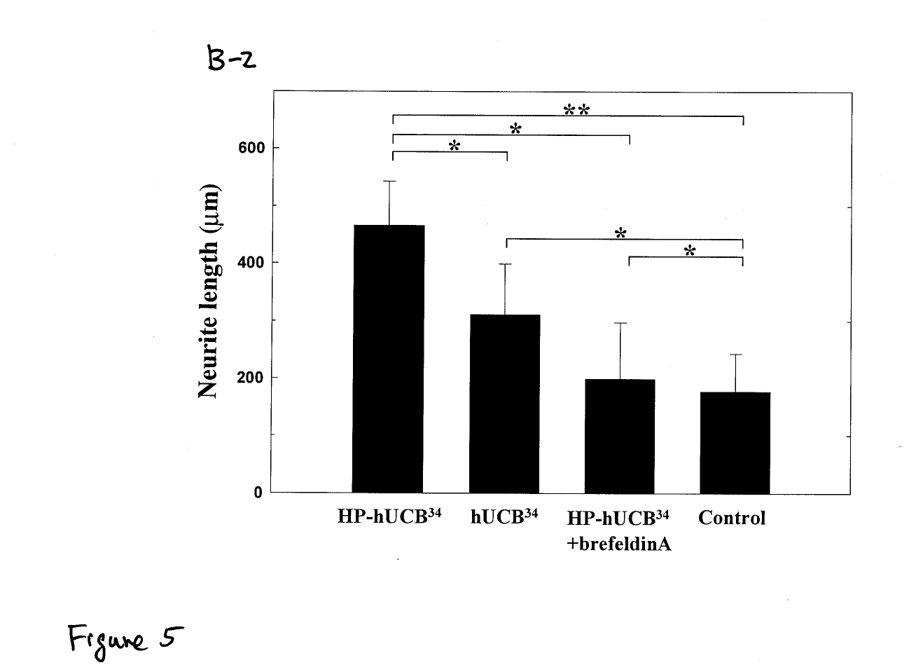

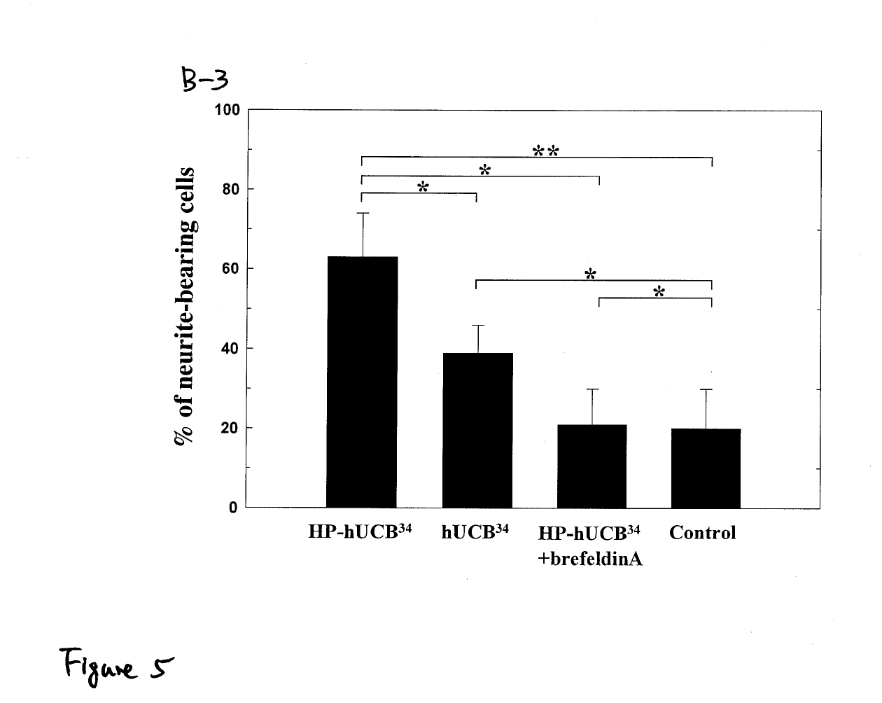

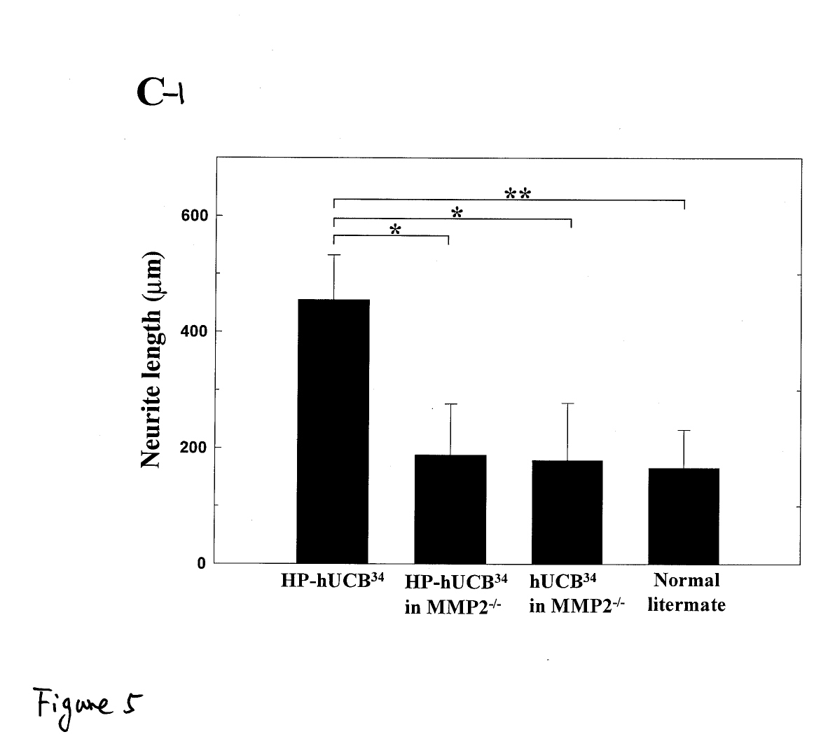

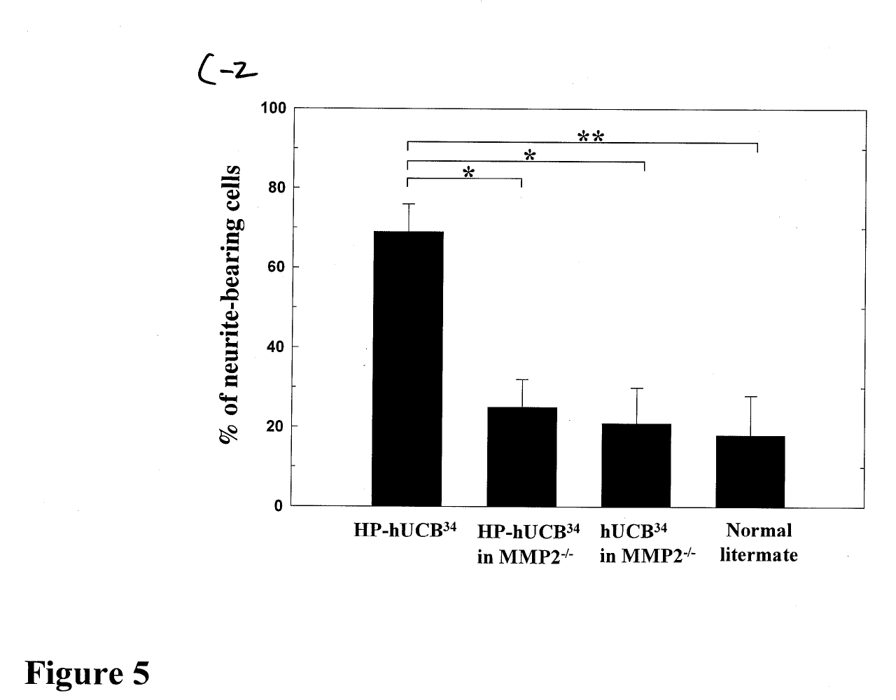

Neurite formation in the stem cells transplantation and control groups was measured to ascertain whether transplantation of HP-hUCB³⁴ or hUCB³⁴ stimulated neurite outgrowth. Intracerebral HP-hUCB³⁴ transplantation significantly improved axonal regeneration in comparison with hUCB³⁴-treated and control rats (FIG. 5B-1). Significantly longer neurites extended over the penumbral areas of HP-hUCB³⁴-treated rats (n=8) than hUCB³⁴-treated (n=8) and control rats (n=8) at 28 days after cerebral ischemia (FIG. 5B-2). Moreover, HP-hUCB³⁴-treated rats (n=8) had more neurite-bearing neurons in the penumbral areas and striatum at 28 days after cerebral ischemia than hUCB³⁴-treated (n=8) and control rats (n=8) (FIG. 5B-3). However, transplantation of brefeldin A-incubated HP-hUCB³⁴ (n=8) could not promote the neurite regeneration in the cerebral ischemic rats (FIGS. 5B-1 to 5B-3). Moreover, HP-hUCB³⁴ and hUCB³⁴ implantation could not significantly improve the neurite degeneration in the MMP2^{-/-} mice (n=8, each) compared with that of their normal litermate mice (n=8) after cerebral ischemia (FIGS. 5C-1 and 5C-2).

The above results demonstrated that HP of CD34-immunosorted human umbilical cord blood hematopoietic stem cells (HP-hUCB³⁴) can activate an exchange protein activated by cAMP (Epac1) through HIF-1a induction. Epac1 activation by HP was indicated by measurement of the expression of Rap1 GTPase-activating protein (Rap1-GTP). Activated Epac1-Rap signaling in HP-hUCB³⁴ promoted neuroplasticity by improving neurological deficit and glucose metabolic activity, and enhanced neural progenitor cells (NPCs) homing in stem cell-implanted cerebral ischemic model. In addition, increasing the activity of MMP2 in HP-hUCB³⁴ through the Epac1-Rap1 cascade also promoted angiogenesis and neurite regeneration in stem cell-implanted stroke rats. In sum, activation of Epac1-Rap1 signaling by HP in hUCB³⁴ further modulate MMP2 activity, which provided a neuroplastic nich in the HP-hUCB³⁴-implanted cerebral ischemic model.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features. From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. A method of improving neurological behavior function of a subject having brain tissue damage, comprising
identifying a subject suffering from brain tissue damage, and
administering to the subject a composition containing an effective amount of a pluripotent cell,
wherein the pluripotent cell is prepared by a process comprising culturing the cell under a hypoxia condition.

2. The method of claim 1, wherein the pluripotent cell is a CD34⁺ cell.

3. The method of claim 1, wherein the pluripotent cell is a CD34⁺ cell and is obtained from umbilical cord blood.

4. The method of claim 1, wherein the process further comprises evaluating the Epac1 level in the cell after culturing the cell under a hypoxia condition.

5. The method of claim 1, wherein the composition is administered intracerebrally.

6. The method of claim 1, wherein the method further comprises evaluating a therapeutic effect on the subject by a non-invasive technique.

7. The method of claim 1, wherein culturing the cell under a hypoxia condition is conducted by placing the cell in a medium containing 60 to 600 mM Desferrioxamine (DFX) for 12 to 48 hours.

8. The method of claim 7, wherein culturing the cell under a hypoxia condition is conducted by placing the cell in a medium containing 100 to 450 mM Desferrioxamine (DFX) for 16 to 36 hours.

9. The method of claim 8, wherein culturing the cell under a hypoxia condition is conducted by placing the cell in a medium containing 200 to 350 mM Desferrioxamine (DFX) for 20 to 24 hours.

10. The method of claim 1, wherein culturing the cell under a hypoxia condition is conducted by placing the cell in an incubator containing 0.5 to 3% O₂ for 6 to 48 hours.

11. The method of claim 10, wherein culturing the cell under a hypoxia condition is conducted by placing the cell in an incubator containing 0.8 to 1.5% O₂ for 12 to 36 hours.

12. The method of claim 11, wherein culturing the cell under a hypoxia condition is conducted by placing the cell in an incubator containing 0.9 to 1.1% O₂ for 23 to 25 hours.

13. A method of increasing angiogenesis in a tissue of a subject, comprising administering to a tissue of a subject in need thereof a composition containing an effective amount of a pluripotent cell, wherein the pluripotent cell is prepared by a process comprising culturing the cell under a hypoxia condition.

14. The method of claim 13, wherein the subject has brain tissue damage and the tissue is the brain of the subject.

15. The method of claim 1, wherein culturing the cell under a hypoxia condition is conducted by placing the cell in a medium containing 10 to 500 μM CoCl₂ for 12 to 48 hours.

16. The method of claim 1, wherein culturing the cell under a hypoxia condition is conducted by placing the cell in a medium containing about 100 μM CoCl₂ for 12 to 48 hours.

17. A composition comprising one or more pluripotent cells and a hypoxia agent.

18. The composition of claim 17, wherein the one or more pluripotent cells are CD34+ cells.

19. The composition of claim 18, wherein the one or more pluripotent cells are obtained from umbilical cord blood.

20. The composition of claim 19, wherein the hypoxia agent is Desferrioxamine or CoCl₂.


SEQUENCE LISTING (2 sequences)

SEQ ID NO: 1 - DNA, 23 bp, Artificial sequence
  Synthetic oligonucleotide
cctcccggccacgtggcggccag

SEQ ID NO: 2 - DNA, 23 bp, Artificial sequence
  Synthetic oligonucleotide
ggagggccggtgcaccgccggtc